SEQUENCE LISTING (106 sequences)

SEQ ID NO: 1 - DNA, 4290 bp, Rattus rattus
  CDS (1)...(4287)
atggaagagaacacgtttggggttcagcagatccaacccaatgtaatttctgttcgtctcttcaaacgcaaagtgggaggtctgggcttcctggtgaaggaacgggtcagcaagcctcccgtgatcatctcagacctgattcgaggaggtgctgcggagcagagcggccttatccaagctggagacatcattctcgcagtcaacgatcggcccttggtagacctcagctatgacagtgccctggaggttctcaggggcattgcctctgagacccacgtggtcctcattctgaggggccctgagggcttcactacacatctggagaccaccttcacaggggatggaacccccaagaccatccgggtgacccagcccctcggtcctcccaccaaagccgtcgatctgtctcaccagccttcagccagcaaagaccagtcattagcagtagacagagtcacaggtctgggtaatggccctcagcatgcccaaggccatgggcagggagctggctcagtctcccaagctaatggtgtggccattgaccccacgatgaaaagcaccaaggccaacctccaggacatcggggaacatgatgaactgctcaaagagatagaacctgtgctgagcatcctcaacagtgggagcaaagccaccaacagagggggaccagccaaagcagagatgaaagacacaggaatccaggtggacagagacctcgatggcaaatcgcacaaagctccgcccctgggcggggacaatgaccgcgtcttcaatgacctgtgggggaaggacaacgttcctgtggtccttaacaacccgtattcagagaaggaacagtcccctacctcggggaaacagtctcccaccaagaacggcagcccttccaggtgcccccgtttcctcaaggtcaagaactgggagacggacgtggtcctcaccgacaccctgcacctgaagagcacactggaaacggggtgcacagagcacatttgcatgggctcgatcatgctgccttcccagcacacgcggaagccagaagatgtccgcacaaaggaccagctcttccctctagccaaagaatttctcgaccaatactactcatccattaagagatttggctccaaggcccacatggacaggctggaggaggtgaacaaggagattgaaagcaccagcacctaccagctcaaggacaccgagctcatctatggcgccaagcatgcctggcggaacgcctctcgatgtgtgggcaggatccagtggtccaagctgcaggtgttcgatgcccgagactgcaccacagcccacggcatgttcaactacatctgtaaccatgtcaagtatgccaccaacaaagggaatctcaggtcggccatcacgatattccctcagaggactgacggcaaacatgacttccgagtgtggaactcgcagctcatccgctacgcgggctacaagcagccagatggctctaccttgggggatccagccaatgtgcagttcacggagatctgtatacagcagggctggaaagccccaagaggccgcttcgacgtgctgcctctcctgcttcaggccaatggcaatgaccctgagctcttccagatccccccagagctggtgctggaagtgcccatcaggcaccccaagttcgactggtttaaggacctggggctcaaatggtatggcctccccgctgtgtccaacatgctgctggagatcgggggcctggagttcagcgcctgtcccttcagcggctggtacatgggcacagagatcggcgtccgtgactactgtgacaactctcgatacaacatcctggaggaagtagccaagaagatggatttggacatgaggaagacctcgtccctctggaaggaccaagcactggtggagatcaacattgctgttctatatagcttccagagtgacaaggtgaccatcgttgaccaccactctgccacggagtccttcatcaaacacatggagaatgaataccgctgcagagggggctgccccgccgactgggtgtggattgtgcctcccatgtcgggcagcatcacccctgtcttccaccaggagatgctcaactatagactcaccccgtcctttgaataccagcctgatccatggaacacccacgtgtggaagggcaccaacgggacccccacgaagcggcgagctatcggctttaagaaattggcagaggccgtcaagttctcagccaagctaatggggcaggccatggccaagagggtcaaggcgaccattctctacgccacagagacaggcaaatcacaagcctatgccaagaccctgtgtgagatcttcaagcacgccttcgatgccaaggcaatgtccatggaggagtatgacatcgtgcacctggagcacgaagccctggtcttggtggtcaccagcacctttggcaatggagacccccctgagaacggggagaaattcggctgtgctttaatggagatgaggcaccccaactctgtgcaggaggagagaaagagctacaaggtccgattcaacagcgtctcctcctattctgactcccgaaagtcatcgggcgacggacccgacctcagagacaactttgaaagtactggacccctggccaatgtgaggttctcagtgttcggcctcggctctcgggcgtacccccacttctgtgcctttgggcatgcggtggacaccctcctggaggaactgggaggggagaggattctgaagatgagggagggggatgagctttgcggacaggaagaagctttcaggacctgggccaagaaagtcttcaaggcagcctgtgatgtgttctgcgtgggggatgacgtcaacatcgagaaggcgaacaactccctcattagcaatgaccgaagctggaagaggaacaagttccgcctcacgtatgtggcggaagctccagatctgacccaaggtctttccaatgttcacaaaaaacgagtctcggctgctcgactcctcagccgccaaaacctgcaaagccctaagtccagccgatcgaccatcttcgtgcgtctccacaccaacgggaatcaggagctgcagtaccagccaggggaccacctgggtgtcttccccggcaaccacgaggacctcgtgaatgcactcattgaacggctggaggatgcaccgcctgccaaccacgtggtgaaggtggagatgctggaggagaggaacactgctctgggtgtcatcagtaattggaaggatgaatctcgcctcccaccctgcaccatcttccaggccttcaagtactacctggacatcaccacgccgcccacgcccctgcagctgcagcagttcgcctctctggccactaatgagaaagagaagcagcggttgctggtcctcagcaaggggctccaggaatatgaggagtggaagtggggcaagaaccccacaatggtggaggtgctggaggagttcccgtccatccagatgccggctacacttctcctcactcagctgtcgctgctgcagcctcgctactactccatcagctcctctccagacatgtaccccgacgaggtgcacctcactgtggccatcgtctcctaccacacccgagacggagaaggaccagtccaccacggggtgtgctcctcctggctcaacagaatacaggctgacgatgtagtcccctgcttcgtgagaggtgcccctagcttccacctgcctcgaaacccccaggtgccttgcatcctggttggcccaggcactggcatcgcacccttccgaagcttctggcaacagcgacaatttgacatccaacacaaaggaatgaatccgtgccccatggttctggtcttcgggtgtcgacaatccaagatagatcatatctacagagaggagaccctgcaggctaagaacaagggcgtcttcagagagctgtacactgcctattcccgggaaccggacaggccaaagaaatatgtacaggacgtgctgcaggaacagctggctgagtctgtgtaccgcgccctgaaggagcaaggaggccacatttatgtctgtggggacgttaccatggccgccgatgtcctcaaagccatccagcgcataatgacccagcaggggaaactctcagaggaggacgctggtgtattcatcagcaggctgagggatgacaaccggtaccacgaggacatctttggagtcaccctcagaacgtatgaagtgaccaaccgccttagatctgagtccatcgccttcatcgaagagagcaaaaaagacgcagatgaggttttcagctcctaa

SEQ ID NO: 2 - protein, 1429 aa, Rattus rattus
MEENTFGVQQIQPNVISVRLFKRKVGGLGFLVKERVSKPPVIISDLIRGGAAEQSGLIQAGDIILAVNDRPLVDLSYDSALEVLRGIASETHVVLILRGPEGFTTHLETTFTGDGTPKTIRVTQPLGPPTKAVDLSHQPSASKDQSLAVDRVTGLGNGPQHAQGHGQGAGSVSQANGVAIDPTMKSTKANLQDIGEHDELLKEIEPVLSILNSGSKATNRGGPAKAEMKDTGIQVDRDLDGKSHKAPPLGGDNDRVFNDLWGKDNVPVVLNNPYSEKEQSPTSGKQSPTKNGSPSRCPRFLKVKNWETDVVLTDTLHLKSTLETGCTEHICMGSIMLPSQHTRKPEDVRTKDQLFPLAKEFLDQYYSSIKRFGSKAHMDRLEEVNKEIESTSTYQLKDTELIYGAKHAWRNASRCVGRIQWSKLQVFDARDCTTAHGMFNYICNHVKYATNKGNLRSAITIFPQRTDGKHDFRVWNSQLIRYAGYKQPDGSTLGDPANVQFTEICIQQGWKAPRGRFDVLPLLLQANGNDPELFQIPPELVLEVPIRHPKFDWFKDLGLKWYGLPAVSNMLLEIGGLEFSACPFSGWYMGTEIGVRDYCDNSRYNILEEVAKKMDLDMRKTSSLWKDQALVEINIAVLYSFQSDKVTIVDHHSATESFIKHMENEYRCRGGCPADWVWIVPPMSGSITPVFHQEMLNYRLTPSFEYQPDPWNTHVWKGTNGTPTKRRAIGFKKLAEAVKFSAKLMGQAMAKRVKATILYATETGKSQAYAKTLCEIFKHAFDAKAMSMEEYDIVHLEHEALVLVVTSTFGNGDPPENGEKFGCALMEMRHPNSVQEERKSYKVRFNSVSSYSDSRKSSGDGPDLRDNFESTGPLANVRFSVFGLGSRAYPHFCAFGHAVDTLLEELGGERILKMREGDELCGQEEAFRTWAKKVFKAACDVFCVGDDVNIEKANNSLISNDRSWKRNKFRLTYVAEAPDLTQGLSNVHKKRVSAARLLSRQNLQSPKSSRSTIFVRLHTNGNQELQYQPGDHLGVFPGNHEDLVNALIERLEDAPPANHVVKVEMLEERNTALGVISNWKDESRLPPCTIFQAFKYYLDITTPPTPLQLQQFASLATNEKEKQRLLVLSKGLQEYEEWKWGKNPTMVEVLEEFPSIQMPATLLLTQLSLLQPRYYSISSSPDMYPDEVHLTVAIVSYHTRDGEGPVHHGVCSSWLNRIQADDVVPCFVRGAPSFHLPRNPQVPCILVGPGTGIAPFRSFWQQRQFDIQHKGMNPCPMVLVFGCRQSKIDHIYREETLQAKNKGVFRELYTAYSREPDRPKKYVQDVLQEQLAESVYRALKEQGGHIYVCGDVTMAADVLKAIQRIMTQQGKLSEEDAGVFISRLRDDNRYHEDIFGVTLRTYEVTNRLRSESIAFIEESKKDADEVFSS

SEQ ID NO: 3 - protein, 10 aa, Artificial Sequence
  nNOS mutated protein fragment
KDTGIQVDWD

SEQ ID NO: 4 - protein, 10 aa, Artificial Sequence
  nNOS mutated protein fragment
IDVGIQVDWD

SEQ ID NO: 5 - protein, 10 aa, Artificial Sequence
  nNOS mutated protein fragment
CDTGIQVDRD

SEQ ID NO: 6 - protein, 10 aa, Artificial Sequence
  nNOS mutated protein fragment
IDTGIQVDRD

SEQ ID NO: 7 - protein, 10 aa, Artificial Sequence
  nNOS mutated protein fragment
VDTGIQVDRD

SEQ ID NO: 8 - protein, 10 aa, Artificial Sequence
  nNOS mutated protein fragment
KDAGIQVDRD

SEQ ID NO: 9 - protein, 10 aa, Artificial Sequence
  nNOS mutated protein fragment
KDCGIQVDRD

SEQ ID NO: 10 - protein, 10 aa, Artificial Sequence
  nNOS mutated protein fragment
KDEGIQVDRD

SEQ ID NO: 11 - protein, 10 aa, Artificial Sequence
  nNOS mutated protein fragment
KDIGIQVDRD

SEQ ID NO: 12 - protein, 10 aa, Artificial Sequence
  nNOS mutated protein fragment
KDKGIQVDRD

SEQ ID NO: 13 - protein, 10 aa, Artificial Sequence
  nNOS mutated protein fragment
KDFGIQVDRD

SEQ ID NO: 14 - protein, 10 aa, Artificial Sequence
  nNOS mutated protein fragment
KDVGIQVDRD

SEQ ID NO: 15 - protein, 10 aa, Artificial Sequence
  nNOS mutated protein fragment
KDTGIQTDRD

SEQ ID NO: 16 - protein, 10 aa, Artificial Sequence
  nNOS mutated protein fragment
KDTGIQVCRD

SEQ ID NO: 17 - protein, 10 aa, Artificial Sequence
  nNOS mutated protein fragment
KDTGIQVNRD

SEQ ID NO: 18 - protein, 10 aa, Artificial Sequence
  nNOS mutated protein fragment
KDTGIQVDLD

SEQ ID NO: 19 - protein, 10 aa, Artificial Sequence
  nNOS mutated protein fragment
KDTGIQVDCD

SEQ ID NO: 20 - protein, 10 aa, Artificial Sequence
  nNOS mutated protein fragment
KDTGIQVDFD

SEQ ID NO: 21 - protein, 10 aa, Artificial Sequence
  nNOS mutated protein fragment
KDTGIQVDYD

SEQ ID NO: 22 - protein, 10 aa, Artificial Sequence
  nNOS mutated protein fragment
KDTGIQVDRF

SEQ ID NO: 23 - protein, 10 aa, Artificial Sequence
  nNOS mutated protein fragment
KDTGIQVDRW

SEQ ID NO: 24 - protein, 10 aa, Artificial Sequence
  nNOS mutated protein fragment
VDTGIQVDRY

SEQ ID NO: 25 - protein, 10 aa, Artificial Sequence
  nNOS mutated protein fragment
IDVGIQVDWW

SEQ ID NO: 26 - protein, 10 aa, Artificial Sequence
  nNOS mutated protein fragment
IDVGIQTDWD

SEQ ID NO: 27 - protein, 10 aa, Artificial Sequence
  nNOS mutated protein fragment
IDVGIQTDWW

SEQ ID NO: 28 - protein, 10 aa, Artificial Sequence
  nNOS mutated protein fragment
IDVGIQTCWW

SEQ ID NO: 29 - protein, 10 aa, Artificial Sequence
  nNOS mutated protein fragment
CDTGIQVDWD

SEQ ID NO: 30 - protein, 10 aa, Artificial Sequence
  nNOS mutated protein fragment
IDTGIQVDWD

SEQ ID NO: 31 - protein, 10 aa, Artificial Sequence
  nNOS mutated protein fragment
VDTGIQVDWD

SEQ ID NO: 32 - protein, 10 aa, Artificial Sequence
  nNOS mutated protein fragment
KDVGIQVDWD

SEQ ID NO: 33 - protein, 10 aa, Artificial Sequence
  nNOS mutated protein fragment
CDVGIQVDWD

SEQ ID NO: 34 - protein, 10 aa, Artificial Sequence
  nNOS mutated protein fragment
VDVGIQVDWD

SEQ ID NO: 35 - protein, 10 aa, Artificial Sequence
  nNOS mutated protein fragment
CDIGIQVDWD

SEQ ID NO: 36 - protein, 10 aa, Artificial Sequence
  nNOS mutated protein fragment
IDIGIQVDWD

SEQ ID NO: 37 - protein, 10 aa, Artificial Sequence
  nNOS mutated protein fragment
VDIGIQVDWD

SEQ ID NO: 38 - protein, 10 aa, Artificial Sequence
  nNOS mutated protein fragment
KDFGIQVDWD

SEQ ID NO: 39 - protein, 10 aa, Artificial Sequence
  nNOS mutated protein fragment
CDFGIQVDWD

SEQ ID NO: 40 - protein, 10 aa, Artificial Sequence
  nNOS mutated protein fragment
IDFGIQVDWD

SEQ ID NO: 41 - protein, 10 aa, Artificial Sequence
  nNOS mutated protein fragment
VDFGIQVDWD

SEQ ID NO: 42 - protein, 10 aa, Artificial Sequence
  nNOS mutated protein fragment
CDEGIQVDWD

SEQ ID NO: 43 - protein, 10 aa, Artificial Sequence
  nNOS mutated protein fragment
IDEGIQVDWD

SEQ ID NO: 44 - protein, 10 aa, Artificial Sequence
  nNOS mutated protein fragment
VDEGIQVDWD

SEQ ID NO: 45 - protein, 10 aa, Artificial Sequence
  nNOS mutated protein fragment
KDCGIQVDWD

SEQ ID NO: 46 - protein, 10 aa, Artificial Sequence
  nNOS mutated protein fragment
CDCGIQVDWD

SEQ ID NO: 47 - protein, 10 aa, Artificial Sequence
  nNOS mutated protein fragment
IDCGIQVDRD

SEQ ID NO: 48 - protein, 10 aa, Artificial Sequence
  nNOS mutated protein fragment
VDCGIQVDRD

SEQ ID NO: 49 - protein, 10 aa, Artificial Sequence
  nNOS mutated protein fragment
HDVGIQVDWD

SEQ ID NO: 50 - protein, 10 aa, Artificial Sequence
  nNOS mutated protein fragment
SDVGIQVDWD

SEQ ID NO: 51 - protein, 10 aa, Artificial Sequence
  nNOS mutated protein fragment
TDVGIQVDWD

SEQ ID NO: 52 - protein, 10 aa, Artificial Sequence
  nNOS mutated protein fragment
KEVGIQVDWD

SEQ ID NO: 53 - protein, 10 aa, Artificial Sequence
  nNOS mutated protein fragment
KDIGIQVDWD

SEQ ID NO: 54 - protein, 10 aa, Artificial Sequence
  nNOS mutated protein fragment
KDEGIQVDWD

SEQ ID NO: 55 - protein, 10 aa, Artificial Sequence
  nNOS mutated protein fragment
KDQGIQVDWD

SEQ ID NO: 56 - protein, 10 aa, Artificial Sequence
  nNOS mutated protein fragment
KDVAIQVDWD

SEQ ID NO: 57 - protein, 10 aa, Artificial Sequence
  nNOS mutated protein fragment
KDVGVQVDWD

SEQ ID NO: 58 - protein, 10 aa, Artificial Sequence
  nNOS mutated protein fragment
KDVGTQVDWD

SEQ ID NO: 59 - protein, 10 aa, Artificial Sequence
  nNOS mutated protein fragment
KDVGIQVDID

SEQ ID NO: 60 - protein, 10 aa, Artificial Sequence
  nNOS mutated protein fragment
KDVGIQVDWE

SEQ ID NO: 61 - protein, 10 aa, Artificial Sequence
  nNOS mutated protein fragment
AIEPVLSILN

SEQ ID NO: 62 - protein, 10 aa, Artificial Sequence
  nNOS mutated protein fragment
RIEPVLSILN

SEQ ID NO: 63 - protein, 10 aa, Artificial Sequence
  nNOS mutated protein fragment
NIEPVLSILN

SEQ ID NO: 64 - protein, 10 aa, Artificial Sequence
  nNOS mutated protein fragment
DIEPVLSILN

SEQ ID NO: 65 - protein, 10 aa, Artificial Sequence
  nNOS mutated protein fragment
QIEPVLSILN

SEQ ID NO: 66 - protein, 10 aa, Artificial Sequence
  nNOS mutated protein fragment
GIEPVLSILN

SEQ ID NO: 67 - protein, 10 aa, Artificial Sequence
  nNOS mutated protein fragment
PIEPVLSILN

SEQ ID NO: 68 - protein, 10 aa, Artificial Sequence
  nNOS mutated protein fragment
SIEPVLSILN

SEQ ID NO: 69 - protein, 10 aa, Artificial Sequence
  nNOS mutated protein fragment
TIEPVLSILN

SEQ ID NO: 70 - protein, 10 aa, Artificial Sequence
  nNOS mutated protein fragment
EFEPVLSILN

SEQ ID NO: 71 - protein, 10 aa, Artificial Sequence
  nNOS mutated protein fragment
EINPVLSILN

SEQ ID NO: 72 - protein, 10 aa, Artificial Sequence
  nNOS mutated protein fragment
EIDPVLSILN

SEQ ID NO: 73 - protein, 10 aa, Artificial Sequence
  nNOS mutated protein fragment
EICPVLSILN

SEQ ID NO: 74 - protein, 10 aa, Artificial Sequence
  nNOS mutated protein fragment
EIQPVLSILN

SEQ ID NO: 75 - protein, 10 aa, Artificial Sequence
  nNOS mutated protein fragment
EIEAVLSILN

SEQ ID NO: 76 - protein, 10 aa, Artificial Sequence
  nNOS mutated protein fragment
EIERVLSILN

SEQ ID NO: 77 - protein, 10 aa, Artificial Sequence
  nNOS mutated protein fragment
EIENVLSILN

SEQ ID NO: 78 - protein, 10 aa, Artificial Sequence
  nNOS mutated protein fragment
EIEDVLSILN

SEQ ID NO: 79 - protein, 10 aa, Artificial Sequence
  nNOS mutated protein fragment
EIEQVLSILN

SEQ ID NO: 80 - protein, 10 aa, Artificial Sequence
  nNOS mutated protein fragment
EIEEVLSILN

SEQ ID NO: 81 - protein, 10 aa, Artificial Sequence
  nNOS mutated protein fragment
EIEGVLSILN

SEQ ID NO: 82 - protein, 10 aa, Artificial Sequence
  nNOS mutated protein fragment
EIEHVLSILN

SEQ ID NO: 83 - protein, 10 aa, Artificial Sequence
  nNOS mutated protein fragment
EIEKVLSILN

SEQ ID NO: 84 - protein, 10 aa, Artificial Sequence
  nNOS mutated protein fragment
EIEMVLSILN

SEQ ID NO: 85 - protein, 10 aa, Artificial Sequence
  nNOS mutated protein fragment
EIESVLSILN

SEQ ID NO: 86 - protein, 10 aa, Artificial Sequence
  nNOS mutated protein fragment
EIETVLSILN

SEQ ID NO: 87 - protein, 10 aa, Artificial Sequence
  nNOS mutated protein fragment
EIEPILSILN

SEQ ID NO: 88 - protein, 10 aa, Artificial Sequence
  nNOS mutated protein fragment
EIEPVPSILN

SEQ ID NO: 89 - protein, 10 aa, Artificial Sequence
  nNOS mutated protein fragment
EIEPVLAILN

SEQ ID NO: 90 - protein, 10 aa, Artificial Sequence
  nNOS mutated protein fragment
EIEPVLVILN

SEQ ID NO: 91 - protein, 10 aa, Artificial Sequence
  nNOS mutated protein fragment
EIEPVLSLLN

SEQ ID NO: 92 - protein, 10 aa, Artificial Sequence
  nNOS mutated protein fragment
EIEPVLSFLN

SEQ ID NO: 93 - protein, 10 aa, Artificial Sequence
  nNOS mutated protein fragment
EIEPVLSWLN

SEQ ID NO: 94 - protein, 10 aa, Artificial Sequence
  nNOS mutated protein fragment
EIEPVLSYLN

SEQ ID NO: 95 - protein, 10 aa, Artificial Sequence
  nNOS mutated protein fragment
EIEPVLSVLN

SEQ ID NO: 96 - protein, 10 aa, Artificial Sequence
  nNOS mutated protein fragment
EIEPVLSILA

SEQ ID NO: 97 - protein, 10 aa, Artificial Sequence
  nNOS mutated protein fragment
EIEPVLSILD

SEQ ID NO: 98 - protein, 10 aa, Artificial Sequence
  nNOS mutated protein fragment
EIEPVLSILQ

SEQ ID NO: 99 - protein, 10 aa, Artificial Sequence
  nNOS mutated protein fragment
EIEPVLSILE

SEQ ID NO: 100 - protein, 10 aa, Artificial Sequence
  nNOS mutated protein fragment
EIEPVLSILG

SEQ ID NO: 101 - protein, 10 aa, Artificial Sequence
  nNOS mutated protein fragment
EIEPVLSILH

SEQ ID NO: 102 - protein, 10 aa, Artificial Sequence
  nNOS mutated protein fragment
EIEPVLSILM

SEQ ID NO: 103 - protein, 10 aa, Artificial Sequence
  nNOS mutated protein fragment
EIEPVLSILP

SEQ ID NO: 104 - protein, 10 aa, Artificial Sequence
  nNOS mutated protein fragment
EIEPVLSILS

SEQ ID NO: 105 - protein, 10 aa, Artificial Sequence
  nNOS mutated protein fragment
EIEPVLSILT

SEQ ID NO: 106 - protein, 10 aa, Artificial Sequence
  nNOS mutated protein fragment
EIEDVLSFLG

(19) United States
(12) Patent Application Publication
(10) Pub. No.: US 2005/0019854 A1
(43) Pub. Date: Jan. 27, 2005

(54) Novel method for screening inhibitors of the linkage between the neuronal nitric oxide synthase associated protein and the protein inhibiting neuronal nitric oxide synthase

(76) Inventors: Rene Gross, Saint Goerges d' Orques (FR); Anne-Dominique Lajoix, Montpellier (FR); Gerard Ribes, Montpellier (FR)

Correspondence Address: CLARK & ELBING LLP, 101 FEDERAL STREET, BOSTON, MA, 02110, US

(21) Appl. No.: 10/475,049

(22) PCT Filed: Apr. 17, 2002
(86) PCT No.: PCT/FR02/01327

(30) Foreign Application Priority Data
Apr. 18, 2001 (FR) 01/05248

(51) Int. Cl. C12N 9/06; C12N 15/85; G01N 33/53

(52) U.S. Cl. 435/69.1; 435/455; 435/191; 435/320.1; 435/325; 435/7.1

(57) ABSTRACT

The invention concerns a detection procedure for compounds modulating the complexation between neuronal nitric oxide synthase protein (nNOS) or one of its variants and the protein inhibitor of neuronal nitric oxide synthase (PIN), in which: a mixture of the said compound, the PIN and the nNOS or one of its variants is incubated, significant variation in the quantity of complex formed between the PIN and the nNOS or one of its variants with respect to a control value is detected, when there is significant variation as defined above, it is concluded that there is binding between the said compound and the PIN, or between the said compound and the nNOS or one of its variants, leading to modulation of the complexation between the PIN and nNOS or one of its variants.

The invention concerns a new screening procedure for inhibitors of the bond between neuronal nitric oxide synthase (nNOS) and the protein inhibitor of neuronal nitric oxide synthase (PIN). The invention also concerns the pancreatic form of nNOS in the rat and all other animal species, including humans, as well as the nucleic acid coding for this protein. The invention also concerns the use of proteins and peptides for the preparation of drugs for the treatment of prediabetic and hyperinsulinic states.

Neuronal nitric oxide synthase protein (NOS) is the enzyme that synthesizes nitric oxide (NO) from its substrate, arginine. It is found in three forms: Two constitutive NOS forms—endothelial NOS (eNOS) and neuronal NOS (nNOS)—and inducible NOS, which is induced by interleukins.

Neuronal NOS was first identified and cloned by D. S. Bredt in rat brain in 1991 (Bredt et al., 1991). In the central nervous system, it plays a role in long-term memory (LTP), and in the peripheral nervous system it is present in the non-adrenergic noncholinergic (NANC) neurons of vessels, the intestine, etc. However, in pathological situations, such as strokes, excess NO production can be harmful and may result in neurotoxicity. Current studies focus more on finding nNOS inhibitors that are capable of blocking the toxic effects of NO on neuronal survival.

S. R. Jaffery and S. H. Snyder (1996) identified an endogenous inhibitor specific to neuronal NOS—the protein inhibitor of neuronal nitric oxide synthase (PIN). This inhibitor interacts with neuronal NOS (nNOS) at amino acids 163 to 245 of nNOS, thereby preventing homodimerization of nNOS and blocking NO production. The PIN protein is thus capable of modulating nNOS activity and preventing any overproduction of NO harmful to cell functioning.

The American patent U.S. Pat. No. 5,908,756 concerns a screening test for compounds that increase or decrease the catalytic activity involved in producing the nitric oxide of the neuronal form of NOS (nNOS). The test compound is added to a mixture of PIN and two nNOS monomers, then catalytic activity is evaluated by measuring its effect on homodimerization of the two nNOS monomers. The test compound is in competition with PIN, since the latter inhibits homodimerization. In short, this test is used to find compounds that modify the catalytic activity of neuronal NO synthase.

Type II diabetes or non-insulin dependent diabetes mellitus (NIDDM) is a heterogeneous disease of multifactoral origin, its development depending on both genetic and environmental factors, including diet, excess weight, lack of exercise and smoking. Associated with the socioeconomic context of industrialized countries, this disease is a major public health problem—it is estimated that 220 million people will be affected by 2007.

Type II diabetes is characterized by two major anomalies, the relative importance of which varies among individuals: A decrease in the ability of insulin to increase glucose uptake by peripheral tissues, i.e. insulin resistance, and a secretory dysfunction in pancreatic β-cells that renders the pancreas unable to secrete sufficient amounts of insulin to compensate for insulin resistance. These two anomalies are preceded by an asymptomatic prodromal period called prediabetes, which can last from several years to several decades. This prediabetic state is not characterized by insulin deficiency, but rather by secretory hyperactivity, which results in hyperinsulinemia.

Hyperinsulinemia is often associated with obesity, a significant risk factor in the development of NIDDM in subjects with a genetic predisposition, especially in certain populations where the incidence of obesity and NIDDM is very high (e.g. the Pima Indians). While hyperinsulinemia often develops to compensate for insulin resistance, secretory hyperactivity and hyperinsulinism are accompanied by high plasma levels of proinsulin and intermediaries in the conversion process. This inappropriate secretion of immature insulin has been found in most studies of subjects presenting with glucose intolerance, i.e. prediabetic subjects. Such insulin secretion constitutes a very poor prognosis—an increase in the proinsulin/insulin ratio is associated with the development of NIDDM and cardiovascular complications within 2-5 years.

The high incidence of NIDDM means it is crucial that innovative drugs be developed to provide patients with a greater range of treatments aimed at correcting pancreatic dysfunction at different stages of the disease, particularly the prediabetic stage and type II diabetes.

Currently, the only drugs used to correct the insulin deficiency are hypoglycemic sulfamides and glinides. However, these insulin-secretory agents cannot be used to correct pancreatic β-cell dysfunction in prediabetic and/or hyperinsulinic patients.

To date, there are no drugs specifically adapted for treating prediabetic and/or hyperinsulinic states.

This invention provides a solution to this problem.

The invention provides, among other things, a new detection procedure for the rapid screening of compounds that decrease the interaction between PIN and nNOS.

The invention also provides a new detection procedure for inhibitors of the interaction between the PIN and nNOS, without modifying the catalytic activity of the neuronal NOS.

Finally, the invention can be used to detect compounds that re-establish the normal insulin response in prediabetic, hyperinsulinic and type II diabetes patients.

The invention concerns a detection procedure for compounds that modulate the complexation between neuronal nitric oxide synthase (nNOS), represented by the sequence SEQ ID NO: 2, or one of its variants, and the protein inhibitor of neuronal nitric oxide synthase (PIN), the modulation of this complexation causing a modification of the insulin response regulated by nNOS or one of its variants, in which:
a mixture of the said compound, the PIN and the nNOS or one of its variants is incubated in conditions that enable the:
   formation of a complex between the PIN and nNOS or one of its variants,
   formation of a complex between the said compound and the PIN, or between the said compound and nNOS or one of its variants;
any significant variation detected in the quantity of complex formed between the PIN and nNOS or one of its variants with respect to a control value corresponds to:
   the quantity of complex formed between the PIN and nNOS or one of its variants in the absence of the test compound, or
   the absence of a complex between the PIN and nNOS or one of its variants, resulting in the absence of PIN or the absence of nNOS or one of its variants, or
   the quantity of complex formed between the PIN and nNOS or one of its variants in the presence of a reference inhibitor; and
when there is significant variation as defined above, it is concluded that there was binding between the said compound and the PIN or between the said compound and the nNOS or one of its variants, leading to modulation of the complexation between the PIN and nNOS or one of its variants.

“Modulation of the complexation between neuronal nitric oxide synthase protein (nNOS) and the protein inhibitor of neuronal nitric oxide synthase (PIN)” means:
a decrease in the quantity of complex formed between the nNOS and PIN under the action of the compound modulating the complexation between nNOS and PIN, this decrease being at least approximately 20%, and preferably approximately 50%, of the quantity of the complex with respect to a control value (100%) corresponding to the absence of the test compound, or
an increase in the quantity of complex formed between the nNOS and PIN under the action of the compound modulating the complexation between nNOS and PIN, this increase being at least approximately 120%, preferably at least approximately 150%, of the quantity of the said complex with respect to the control value (100%) corresponding to the absence of the test compound.

“The nNOS or one of its variants” means all neuronal NOS in all species, particularly humans, expressed in different tissues that could present point mutations, specific alternative gene splicing, or both.

“Modification of the insulin response” means a decrease in the insulin response under the action of the compound modulating the complexation between nNOS and PIN, i.e. an increase in the insulin response under the effect of the test compound.

The modification of the insulin response induced by the compound modulating the complexation between the nNOS and PIN can be measured by cell tests, notably by using the INS-1 cell line (Asfari et al., 1992) under one of two conditions:
if the test compound is liposoluble and therefore able to pass through cell membranes, the cells are stimulated using increasing concentrations of glucose (0.5, 1.5 and 2 g/L) in the presence of the test compound in albuminated Krebs-Ringer buffer (Asfari et al., 1992);
if the test compound is not liposoluble and therefore cannot pass through cell membranes, the cells are permeabilized in the presence of Staphylococcus aureus α-toxin (Maechler et al., 1997) and stimulated with an insulin secretory compound (active in the cells) in the presence of the test compound (Maechler et al., 1997).

“Significant variation in the complex formed between the PIN and nNOS or one of its variants”, means an increase or decrease of at least 20%, preferably 50%, in the quantity of complex formed under the action of the test compound with respect to a control value corresponding to the absence of the test compound.

To quantify the complexation rate between the PIN and nNOS, the invention uses a detection procedure involving the molecular marking of one of the binding partners, i.e. the PIN and/or nNOS, with a substance such as a radioactive, fluorescent or luminescent element, an enzyme or a biotin. This marking provides a direct or indirect quantitative physical measurement based on emissions (radioactive, luminescent or fluorescent rays) or signal absorption (light or fluorescence), either spontaneously or after addition of an enzymatic substrate or light excitation. To quantify the complexation rate between the PIN and nNOS, surface plasmon resonance (e.g. Biacore AB), which does not entail marking of one of the two partners, can also be used, provided the PIN or nNOS is immobilized in the biosensor channel. To quantify the complexation rate between the PIN and nNOS, two-hybrid assays on yeast or bacteria can also be used, as described in U.S. Pat. Nos. 5,283,173 and 5,468,614. This technique, which is done in vivo, does not require that the PIN and nNOS be produced and purified.

In one method described in the invention, the quantity of complex formed (or its variation) can de determined in solution if the PIN and nNOS are marked with substances capable of energy exchange. When the complex forms, the two binding partners move closer together, leading to a transfer of energy between the two markers and an increase or decrease in the intensity of the fluorescent signal emitted by one of the two markers.

In another method, the quantity of complex (or its variation) can be determined in solid phase if one of the two binding partners is immobilized on a solid substrate. The bond of the other binding partner is detected by surface plasmon resonance or by marking this partner as per the detection system defined above.

The first binding partner, i.e. the nNOS or PIN, can be immobilized either covalently (chemical reaction) or non-covalently by physicochemical interactions (adsorption on a hydrophobic plastic surface), or by biospecific interactions in which the biological sensor is first immobilized on a plate (antibodies specific to the first binding partner, or avidin if the first binding partner is coated in biotin).

The second binding partner, i.e. the PIN or nNOS, is used to determine the quantity of complex formed (or its variation), either directly by surface plasmon resonance, or indirectly by quantitative detection of its marker. The marker can be either an element attached to the second partner by chemical bonding (radioactive, fluorescent or luminescent element, enzyme, biotin), some other biospecific substance, such as an antibody against the other binding partner (itself marked either directly or indirectly), or avidin (marked directly or indirectly).

The control value can be obtained in one of the following ways:
running an experiment in which a mixture of PIN and nNOS or one of its variants is incubated in conditions leading to the formation of a complex between PIN and nNOS, then determining the quantity of complex formed between PIN and nNOS, this quantity being the control value;
running an experiment in which only the PIN is incubated, leading to no complex formation between the nNOS and PIN,
running an experiment in which only the nNOS protein is incubated, leading to no complex formation between the nNOS and PIN,
running an experiment in which a mixture of the PIN, the nNOS or one of its variants and a reference inhibitor is incubated in conditions leading to the formation of a complex between the PIN and nNOS or one of its variants, and detection of the quantity of complex formed between the PIN and nNOS, this quantity being the control value,
running an experiment in which the PIN, preincubated with the reference inhibitor, is added to the nNOS which has been immobilized on a biosensor (e.g. Biacore AB), and the quantity of complex formed between the PIN and nNOS determined, e.g. by surface plasmon resonance, this quantity being the control value.

The reference inhibitor can be chosen, for example, from among the peptides represented by sequences SEQ ID NO: 3 and SEQ ID NO: 4.

The complex formed between the PIN and nNOS can be detected using the techniques defined above.

This test is used to target the early stages of type II diabetes, hyperinsulinic states and overt type II diabetes by using the insulin-modulating properties of the pancreatic form of NOS and its endogenous inhibitor, PIN.

The invention also concerns a detection procedure as defined above, in which the compound is characterized as not substantially modifying the catalytic activity of the nNOS or one of its variants.

“Without substantially modifying the catalytic activity of the nNOS or one of its variants” means the test compound is only able to slightly affect NO production activity of the nNOS.

There are two possible scenarios:
no modification of catalytic activity if the said compound binds only to the PIN protein, and
no modification or an increase or decrease of no more than 20-30% of the base catalytic activity of the nNOS in the absence of the said compound, if the said compound binds to the nNOS.

The catalytic activity of the nNOS can be estimated, for example, by its ability to produce radiolabelled citrulline from its substrate, radiolabelled arginine, and in the presence of cofactors such as BH₄, FAD, FMN, NADPH, Ca²⁺ and calmodulin. The citrulline produced can be separated from the arginine by ion-exchange chromatography and quantified by a radioactivity count.

The invention concerns a detection procedure for compounds that decrease the complexation between neuronal nitric oxide synthase (nNOS) or one of its variants, and the protein inhibitor of neuronal nitric oxide synthase (PIN), the decrease in this complexation leading to a reduction in the insulin response regulated by the nNOS or one of its variants, in which:
a mixture of the said compound, the PIN and the nNOS or one of its variants is incubated in conditions that enable the:
   formation of a complex between the PIN and nNOS or one of its variants,
   formation of a complex between the said compound and the PIN, or between the said compound and the nNOS or one of its variants;
any significant decrease detected in the quantity of complex formed between the PIN and nNOS or one of its variants with respect to a control value corresponds to:
   the quantity of complex formed between the PIN and nNOS or one of its variants in the absence of the compound submitted to the detection procedure, or
   the absence of complex formed between the PIN and nNOS or one of its variants, resulting in the absence of PIN or the absence of nNOS or one of its variants, or
   the quantity of complex formed between the PIN and nNOS or one of its variants in the presence of a reference inhibitor; and
when there is significant decrease as defined above, it is concluded that there was binding between the said compound and the PIN, or between the said compound and the nNOS or one of its variants, leading to reduction of the complexation between the PIN and nNOS or one of its variants.

“Reduction of the insulin response” means a decrease of at least approximately 20%, and preferably at least approximately 50%, in insulin secretion under the action of the compound decreasing complexation between the nNOS and PIN, with respect to the control value corresponding to the absence of the test compound.

“Significant decrease in the quantity of complex formed between the PIN and nNOS or one of its variants” means a variation of at least approximately 20%, and preferably at least approximately 50%, in the quantity of complex formed in the presence of the test compound, with respect to a control value corresponding to the absence of the test compound.

The invention also concerns a detection procedure for compounds that increase the complexation between neuronal nitric oxide synthase (nNOS) or one of its variants, and the protein inhibitor of neuronal nitric oxide synthase (PIN), the increase in this complexation leading to an amplification of the insulin response regulated by the nNOS or one of its variants, in which:
a mixture of the said compound, the PIN and the nNOS or one of its variants is incubated in conditions that enable the:
   formation of a complex between the PIN and nNOS or one of its variants,
   formation of a complex between the said compound and the PIN, between the said compound and the nNOS or one of its variants;
any significant increase detected in the quantity of complex formed between the PIN and nNOS or one of its variants with respect to a control value corresponds to:
   the quantity of complex formed between the PIN and nNOS or one of its variants in the absence of test compound submitted to the detection procedure, or
   the absence of complex formed between the PIN and nNOS or one of its variants, resulting in the absence of PIN or the absence of nNOS or one of its variants, or
   the quantity of complex formed between the PIN and nNOS or one of its variants in the presence of a reference inhibitor; and
when there is a significant increase as defined above, it is concluded that there was binding between the said compound and the PIN, or between the said compound and the nNOS or one of its variants, leading to an increase in complexation between the PIN and nNOS or one of its variants.

“Amplification of the insulin response” means an increase of at least approximately 20%, and preferably at least approximately 50%, in insulin secretion under the action of the compound increasing complexation between nNOS and PIN, with respect to the control value corresponding to the absence of the test compound.

The invention concerns a detection procedure, as defined above, in which the nNOS protein used is either the pancreatic form of nNOS or the form present in the brain.

The rat nNOS protein present in the pancreatic cells is pancreatic nNOS. It is the mutated form of rat neuronal NOS.

The rat pancreatic nNOS has three amino acid mutations with respect to the nNOS of the rat brain. While these mutations are not localized to the enzyme's functional domains (linkage regions for cofactors such as mentioned above), or to the interaction region between PIN and nNOS, they do affect its tri-dimensional conformation and thus confer on it pancreatic specificity.

The nNOS protein used can also be rat nNOS, which is present in rat brain.

A good detection procedure to use, according to the invention, is the one defined above that involves detecting variation, i.e. any significant decrease in the quantity of complex formed between the PIN and nNOS, with respect to three control values, the first control value corresponding to the quantity of complex formed between the PIN and nNOS in the absence of the compound submitted to the detection procedure; the second control value corresponding to the absence of the complex between the PIN and nNOS, resulting from the absence of PIN or the absence of nNOS; and the third control value corresponding to the quantity of complex formed between the PIN and nNOS in the presence of a reference inhibitor.

The quantity of complex formed between the PIN and nNOS or one of its variants can be detected using one of the techniques defined above.

The first control value can be obtained, for example, by running the following experiment:
incubating a mixture of the PIN and nNOS or one of its variants in conditions leading to the formation of a complex between the PIN and nNOS or one of its variants,
determining the quantity of complex formed between the PIN and nNOS or one of its variants, this quantity being the control value.

The second control value can be obtained, for example, by running one of the following experiments:
incubating only the PIN, which leads to no complex formation between the nNOS or one of its variants and the PIN,
incubating only the nNOS or one of its variants, which leads to no complex formation between the nNOS and one of its variants and the PIN.

The third control value can be obtained, for example, by running the following experiment:
incubating a mixture of the PIN, the nNOS or one of its variants and the reference inhibitor,
detecting the quantity of complex formed between the PIN and nNOS or one of its variants, this quantity being the control value.

The third control value can also be obtained by running the following experiment:
adding the PIN, preincubated with the reference inhibitor, to the nNOS that has been immobilized on a biosensor (e.g. Biacore AB),
determining, i.e. by surface plasmon resonance, the quantity of complex formed between the PIN and nNOS or one of its variants, this quantity being the control value.

A good detection procedure to use, according to the invention, is the one defined above, in which the mixture of the PIN, the nNOS and the test compound is prepared in one of the following ways:
simultaneously adding the PIN, the nNOS and the compound submitted to the detection procedure,
consecutively adding the PIN, the compound submitted to the detection procedure and the nNOS, or adding, consecutively the nNOS, the compound submitted to the detection procedure and the PIN,
adding the said compound previously incubated with the PIN or the nNOS, followed by either the nNOS or PIN, respectively.

When the mixture of the PIN, the nNOS and the compound submitted to the detection procedure is prepared by simultaneously adding the PIN, the nNOS and the said compound, it is easier to detect compounds that bind to the complex formed between the PIN and nNOS, and that dissociate the said complex, as well as compounds that bind only to one of the two binding partners, i.e. the PIN or nNOS, and are effective enough to compete with the complexation.

When the mixture of the PIN, the nNOS and the compound submitted to the detection procedure is prepared by consecutively adding the PIN, the said compound and the nNOS, it is easier to detect compounds that bind to the PIN, as well as locate good ligands of the nNOS (ligands with a strong affinity for nNOS, in the order of μM, preferably nM), which are kinetically limited in this case.

When the mixture of the PIN, nNOS and the compound submitted to the detection process is prepared by consecutively adding the nNOS, the said compound and the PIN, it is easier to detect compounds that bind to the nNOS, as well as locate good ligands of the PIN (ligands with a strong affinity for PIN, in the order of μM, preferably nM), which are kinetically limited in this case.

When the mixture of the PIN, the nNOS and the compound submitted to the detection procedure is prepared by adding the said compound, previously incubated with the PIN, to the nNOS, bonding between the said compound and the PIN is facilitated before the nNOS is added, thereby making it easier to detect compounds bound to the PIN.

When the mixture of the PIN, the nNOS and the compound submitted to the detection procedure is prepared by adding the said compound, previously incubated with the nNOS, to the PIN, bonding between the said compound and the nNOS is facilitated before the PIN is added, thereby making it easier to detect compounds bound to the nNOS.

When the PIN, the nNOS and the said compound are added simultaneously, the quantification of complexes formed can be done in solution using fluorescent markers and fluorescence polarization (or transfer). This procedure has the advantage of being rapid (only one incubation step, no washes) and of being a direct detection system.

When the PIN, the said compound and the nNOS are added consecutively, or after preincubation of the PIN or nNOS with the said compound, one of the two binding partners must first be immobilized on a solid substrate. The quantification of complexes formed is done by surface plasmon resonance or by marking of the other partner. This procedure makes it possible to determine the partner to which the compound binds, i.e. the PIN, the nNOS or the complex formed between the two proteins.

A good detection procedure to use is the one defined above, in which the nNOS is first fixed on a solid substrate.

“Fixed on a solid substrate” refers to a procedure in which the nNOS is immobilized covalently (chemical reaction) or non-covalently (non-specific adsorption on plastic, avidin-biotin system, antibodies) on a solid substrate.

When the nNOS is first fixed on a solid substrate, the PIN bond is detected by surface plasmon resonance or by marking with a detection system (radioactive, luminescent or fluorescent marker, enzyme, biotin), which enables the quantity of complex formed to be measured.

When the nNOS is first fixed on a solid substrate, the simultaneous addition of the PIN and the compound submitted to the detection procedure, not previously mixed, makes it possible to detect ligands of the nNOS, the PIN and the complex formed between the two proteins. This procedure therefore makes it easier to detect molecules that inhibit the association between PIN and nNOS, and thus screen for compounds that dissociate the complex formed between the two proteins.

When the nNOS is first fixed on a solid substrate, the consecutive addition of the compound submitted to the detection procedure and the PIN makes it possible to detect compounds that inhibit only the nNOS. In effect, if the test compound does not bind to the nNOS, it is eliminated during the washing that takes place before addition of the PIN.

When the nNOS is first fixed on a solid substrate, the addition of the compound submitted to the detection procedure, previously incubated with the PIN, makes it possible to detect the ligands of the PIN, the nNOS and the complex formed between the PIN and nNOS. This method facilitates the binding of the said compound with the PIN before incubation with the nNOS, thereby making it possible to screen for ligands of the PIN. This method also makes it possible to select good ligands of the nNOS (ligands with a very strong affinity for nNOS, in the order of μM, preferably nM), which are kinetically limited in this case.

The invention also concerns a detection procedure as defined above, in which the PIN is first fixed on a solid substrate.

“Fixed on a solid substrate” refers to a procedure in which the PIN is immobilized covalently (chemical reaction) or non-covalently (non-specific adsorption on plastic, avidin-biotin system, antibodies) on a solid substrate.

When the PIN is first fixed on a solid substrate, the nNOS bond is detected by surface plasmon resonance or by marking with a detection system (radioactive, luminescent or fluorescent marker, enzyme, biotin), which enables the quantity of complex formed to be measured.

When the PIN is first fixed on a solid substrate, the simultaneous addition of the nNOS and the compound submitted to the detection procedure, not previously mixed, makes it possible to detect ligands of the nNOS, the PIN and the complex formed between the two proteins. This procedure therefore makes it easier to detect molecules that inhibit the association between PIN and nNOS, and thus screen for compounds that dissociate the complex formed between the two proteins.

When the PIN protein is first fixed on a solid substrate, the consecutive addition of the compound submitted to the detection procedure and the nNOS makes it possible to detect compounds that inhibit only the PIN. In effect, if the test compound does not bind to the PIN, it is eliminated during the washing that takes place before addition of the nNOS.

When the PIN is first fixed on a solid substrate, the addition of the compound submitted to the detection procedure, previously incubated with the nNOS, makes it possible to detect the ligands of the PIN, the nNOS and the complex formed between the PIN and nNOS. This method facilitates the binding of the said compound with the nNOS before incubation with the PIN, thereby making it possible to screen for ligands of the nNOS. This method also makes it possible to select good ligands of the PIN (ligands with a very strong affinity for the PIN, in the order of μM, preferably nM), which are kinetically limited in this case.

A good detection procedure to use, according to the invention, is the one defined above, in which the PIN and nNOS are in solution.

When the PIN and nNOS are in solution, the quantification of complexes formed is done by marking the two proteins with a fluorescent compound and measuring fluorescence polarization.

The invention also concerns a protein characterized in that it comprises or is constituted from the sequence SEQ ID NO: 2, or a fragment of the said protein containing at least 100 amino acids, provided the said fragment contains the amino acid in position 269.

SEQ ID NO: 2 is a new protein isolated from the rat that corresponds to the pancreatic form of neuronal nNOS.

The pancreatic form of nNOS is a mutated form of nNOS. It has four nucleotide mutations in positions 269, 953, 1008 and 1299.

The last of these mutations is a silent mutation; therefore, it does not cause a change in amino acid.

The invention concerns the following peptides sequences:

| | |
|---|---|
| Lys-Asp-Thr-Gly-Ile-Gln-Val-Asp- | (SEQ ID NO: 3) |
| Trp-Asp, | |
| Ile-Asp-Val-Gly-Ile-Gln-Val-Asp- | (SEQ ID NO: 4) |
| Trp-Asp, | |
| Cys-Asp-Thr-Gly-Ile-Gln-Val-Asp- | (SEQ ID NO: 5) |
| Arg-Asp, | |
| Ile-Asp-Thr-Gly-Ile-Gln-Val-Asp- | (SEQ ID NO: 6) |
| Arg-Asp, | |
| Val-Asp-Thr-Gly-Ile-Gln-Val-Asp- | (SEQ ID NO: 7) |
| Arg-Asp, | |
| Lys-Asp-Ala-Gly-Ile-Gln-Val-Asp- | (SEQ ID NO: 8) |
| Arg-Asp, | |
| Lys-Asp-Cys-Gly-Ile-Gln-Val-Asp- | (SEQ ID NO: 9) |
| Arg-Asp, | |
| Lys-Asp-Glu-Gly-Ile-Gln-Val-Asp- | (SEQ ID NO: 10) |
| Arg-Asp, | |
| Lys-Asp-Ile-Gly-Ile-Gln-Val-Asp- | (SEQ ID NO: 11) |
| Arg-Asp, | |
| Lys-Asp-Lys-Gly-Ile-Gln-Val-Asp- | (SEQ ID NO: 12) |
| Arg-Asp, | |
| Lys-Asp-Phe-Gly-Ile-Gln-Val-Asp- | (SEQ ID NO: 13) |
| Arg-Asp, | |
| Lys-Asp-Val-Gly-Ile-Gln-Val-Asp- | (SEQ ID NO: 14) |
| Arg-Asp, | |
| Lys-Asp-Thr-Gly-Ile-Gln-Thr-Asp- | (SEQ ID NO: 15) |
| Arg-Asp, | |
| Lys-Asp-Thr-Gly-Ile-Gln-Val-Cys- | (SEQ ID NO: 16) |
| Arg-Asp, | |
| Lys-Asp-Thr-Gly-Ile-Gln-Val-Asn- | (SEQ ID NO: 17) |
| Arg-Asp, | |
| Lys-Asp-Thr-Gly-Ile-Gln-Val-Asp- | (SEQ ID NO: 18) |
| Leu-Asp, | |
| Lys-Asp-Thr-Gly-Ile-Gln-Val-Asp- | (SEQ ID NO: 19) |
| Cys-Asp, | |
| Lys-Asp-Thr-Gly-Ile-Gln-Val-Asp- | (SEQ ID NO: 20) |
| Phe-Asp, | |
| Lys-Asp-Thr-Gly-Ile-Gln-Val-Asp- | (SEQ ID NO: 21) |
| Tyr-Asp, | |
| Lys-Asp-Thr-Gly-Ile-Gln-Val-Asp- | (SEQ ID NO: 22) |
| Arg-Phe, | |
| Lys-Asp-Thr-Gly-Ile-Gln-Val-Asp- | (SEQ ID NO: 23) |
| Arg-Trp, | |
| Val-Asp-Thr-Gly-Ile-Gln-Val-Asp- | (SEQ ID NO: 24) |
| Arg-Tyr, | |
| Ile-Asp-Val-Gly-Ile-Gln-Val-Asp- | (SEQ ID NO: 25) |
| Trp-Trp, | |
| Ile-Asp-Val-Gly-Ile-Gln-Thr-Asp- | (SEQ ID NO: 26) |
| Trp-Asp, | |
| Ile-Asp-Val-Gly-Ile-Gln-Thr-Asp- | (SEQ ID NO: 27) |
| Trp-Trp, | |
| Ile-Asp-Val-Gly-Ile-Gln-Thr-Cys- | (SEQ ID NO: 28) |
| Trp-Trp, | |
| Cys-Asp-Thr-Gly-Ile-Gln-Val-Asp- | (SEQ ID NO: 29) |
| Trp-Asp, | |
| Ile-Asp-Thr-Gly-Ile-Gln-Val-Asp- | (SEQ ID NO: 30) |
| Trp-Asp, | |
| Val-Asp-Thr-Gly-Ile-Gln-Val-Asp- | (SEQ ID NO: 31) |
| Trp-Asp, | |
| Lys-Asp-Val-Gly-Ile-Gln-Val-Asp- | (SEQ ID NO: 32) |
| Trp-Asp, | |
| Cys-Asp-Val-Gly-Ile-Gln-Val-Asp- | (SEQ ID NO: 33) |
| Trp-Asp, | |
| Val-Asp-Val-Gly-Ile-Gln-Val-Asp- | (SEQ ID NO: 34) |
| Trp-Asp, | |
| Cys-Asp-Ile-Gly-Ile-Gln-Val-Asp- | (SEQ ID NO: 35) |
| Trp-Asp, | |
| Ile-Asp-Ile-Gly-Ile-Gln-Val-Asp- | (SEQ ID NO: 36) |
| Trp-Asp, | |
| Val-Asp-Ile-Gly-Ile-Gln-Val-Asp- | (SEQ ID NO: 37) |
| Trp-Asp, | |
| Lys-Asp-Phe-Gly-Ile-Gln-Val-Asp- | (SEQ ID NO: 38) |
| Trp-Asp, | |
| Cys-Asp-Phe-Gly-Ile-Gln-Val-Asp- | (SEQ ID NO: 39) |
| Trp-Asp, | |
| Ile-Asp-Phe-Gly-Ile-Gln-Val-Asp- | (SEQ ID NO: 40) |
| Trp-Asp, | |
| Val-Asp-Phe-Gly-Ile-Gln-Val-Asp- | (SEQ ID NO: 41) |
| Trp-Asp, | |
| Cys-Asp-Glu-Gly-Ile-Gln-Val-Asp- | (SEQ ID NO: 42) |
| Trp-Asp, | |
| Ile-Asp-Glu-Gly-Ile-Gln-Val-Asp- | (SEQ ID NO: 43) |
| Trp-Asp, | |
| Val-Asp-Glu-Gly-Ile-Gln-Val-Asp- | (SEQ ID NO: 44) |
| Trp-Asp, | |
| Lys-Asp-Cys-Gly-Ile-Gln-Val-Asp- | (SEQ ID NO: 45) |
| Trp-Asp, | |
| Cys-Asp-Cys-Gly-Ile-Gln-Val-Asp- | (SEQ ID NO: 46) |
| Trp-Asp, | |
| Ile-Asp-Cys-Gly-Ile-Gln-Val-Asp- | (SEQ ID NO: 47) |
| Arg-Asp, | |
| Val-Asp-Cys-Gly-Ile-Gln-Val-Asp- | (SEQ ID NO: 48) |
| Arg-Asp, | |
| His-Asp-Val-Gly-Ile-Gln-Val-Asp- | (SEQ ID NO: 49) |
| Trp-Asp, | |
| Ser-Asp-Val-Gly-Ile-Gln-Val-Asp- | (SEQ ID NO: 50) |
| Trp-Asp, | |
| Thr-Asp-Val-Gly-Ile-Gln-Val-Asp- | (SEQ ID NO: 51) |
| Trp-Asp, | |
| Lys-Glu-Val-Gly-Ile-Gln-Val-Asp- | (SEQ ID NO: 52) |
| Trp-Asp, | |
| Lys-Asp-Ile-Gly-Ile-Gln-Val-Asp- | (SEQ ID NO: 53) |
| Trp-Asp, | |
| Lys-Asp-Glu-Gly-Ile-Gln-Val-Asp- | (SEQ ID NO: 54) |
| Trp-Asp, | |
| Lys-Asp-Gln-Gly-Ile-Gln-Val-Asp- | (SEQ ID NO: 55) |
| Trp-Asp, | |
| Lys-Asp-Val-Ala-Ile-Gln-Val-Asp- | (SEQ ID NO: 56) |
| Trp-Asp, | |
| Lys-Asp-Val-Gly-Val-Gln-Val-Asp- | (SEQ ID NO: 57) |
| Trp-Asp, | |
| Lys-Asp-Val-Gly-Thr-Gln-Val-Asp- | (SEQ ID NO: 58) |
| Trp-Asp, | |
| Lys-Asp-Val-Gly-Ile-Gln-Val-Asp- | (SEQ ID NO: 59) |
| Ile-Asp, | |
| Lys-Asp-Val-Gly-Ile-Gln-Val-Asp- | (SEQ ID NO: 60) |
| Trp-Glu, | |
| Ala-Ile-Glu-Pro-Val-Leu-Ser-Ile- | (SEQ ID NO: 61) |
| Leu-Asn, | |
| Arg-Ile-Glu-Pro-Val-Leu-Ser-Ile- | (SEQ ID NO: 62) |
| Leu-Asn, | |
| Asn-Ile-Glu-Pro-Val-Leu-Ser-Ile- | (SEQ ID NO: 63) |
| Leu-Asn, | |
| Asp-Ile-Glu-Pro-Val-Leu-Ser-Ile- | (SEQ ID NO: 64) |
| Leu-Asn, | |
| Gln-Ile-Glu-Pro-Val-Leu-Ser-Ile- | (SEQ ID NO: 65) |
| Leu-Asn, | |
| Gly-Ile-Glu-Pro-Val-Leu-Ser-Ile- | (SEQ ID NO: 66) |
| Leu-Asn, | |
| Pro-Ile-Glu-Pro-Val-Leu-Ser-Ile- | (SEQ ID NO: 67) |
| Leu-Asn, | |
| Ser-Ile-Glu-Pro-Val-Leu-Ser-Ile- | (SEQ ID NO: 68) |
| Leu-Asn, | |
| Thr-Ile-Glu-Pro-Val-Leu-Ser-Ile- | (SEQ ID NO: 69) |
| Leu-Asn, | |
| Glu-Phe-Glu-Pro-Val-Leu-Ser-Ile- | (SEQ ID NO: 70) |
| Leu-Asn, | |
| Glu-Ile-Asn-Pro-Val-Leu-Ser-Ile- | (SEQ ID NO: 71) |
| Leu-Asn, | |
| Glu-Ile-Asp-Pro-Val-Leu-Ser-Ile- | (SEQ ID NO: 72) |
| Leu-Asn, | |
| Glu-Ile-Cys-Pro-Val-Leu-Ser-Ile- | (SEQ ID NO: 73) |
| Leu-Asn, | |
| Glu-Ile-Gln-Pro-Val-Leu-Ser-Ile- | (SEQ ID NO: 74) |
| Leu-Asn, | |
| Glu-Ile-Glu-Ala-Val-Leu-Ser-Ile- | (SEQ ID NO: 75) |
| Leu-Asn, | |
| Glu-Ile-Glu-Arg-Val-Leu-Ser-Ile- | (SEQ ID NO: 76) |
| Leu-Asn, | |
| Glu-Ile-Glu-Asn-Val-Leu-Ser-Ile- | (SEQ ID NO: 77) |
| Leu-Asn, | |
| Glu-Ile-Glu-Asp-Val-Leu-Ser-Ile- | (SEQ ID NO: 78) |
| Leu-Asn, | |
| Glu-Ile-Glu-Gln-Val-Leu-Ser-Ile- | (SEQ ID NO: 79) |
| Leu-Asn, | |
| Glu-Ile-Glu-Glu-Val-Leu-Ser-Ile- | (SEQ ID NO: 80) |
| Leu-Asn, | |
| Glu-Ile-Glu-Gly-Val-Leu-Ser-Ile- | (SEQ ID NO: 81) |
| Leu-Asn, | |
| Glu-Ile-Glu-His-Val-Leu-Ser-Ile- | (SEQ ID NO: 82) |
| Leu-Asn, | |
| Glu-Ile-Glu-Lys-Val-Leu-Ser-Ile- | (SEQ ID NO: 83) |
| Leu-Asn, | |
| Glu-Ile-Glu-Met-Val-Leu-Ser-Ile- | (SEQ ID NO: 84) |
| Leu-Asn, | |
| Glu-Ile-Glu-Ser-Val-Leu-Ser-Ile- | (SEQ ID NO: 85) |
| Leu-Asn, | |
| Glu-Ile-Glu-Thr-Val-Leu-Ser-Ile- | (SEQ ID NO: 86) |
| Leu-Asn, | |
| Glu-Ile-Glu-Pro-Ile-Leu-Ser-Ile- | (SEQ ID NO: 87) |
| Leu-Asn, | |
| Glu-Ile-Glu-Pro-Val-Pro-Ser-Ile- | (SEQ ID NO: 88) |
| Leu-Asn, | |
| Glu-Ile-Glu-Pro-Val-Leu-Ala-Ile- | (SEQ ID NO: 89) |
| Leu-Asn, | |
| Glu-Ile-Glu-Pro-Val-Leu-Val-Ile- | (SEQ ID NO: 90) |
| Leu-Asn, | |
| Glu-Ile-Glu-Pro-Val-Leu-Ser-Leu- | (SEQ ID NO: 91) |
| Leu-Asn, | |
| Glu-Ile-Glu-Pro-Val-Leu-Ser-Phe- | (SEQ ID NO: 92) |
| Leu-Asn, | |
| Glu-Ile-Glu-Pro-Val-Leu-Ser-Trp- | (SEQ ID NO: 93) |
| Leu-Asn, | |
| Glu-Ile-Glu-Pro-Val-Leu-Ser-Tyr- | (SEQ ID NO: 94) |
| Leu-Asn, | |
| Glu-Ile-Glu-Pro-Val-Leu-Ser-Val- | (SEQ ID NO: 95) |
| Leu-Asn, | |
| Glu-Ile-Glu-Pro-Val-Leu-Ser-Ile- | (SEQ ID NO: 96) |
| Leu-Ala, | |
| Glu-Ile-Glu-Pro-Val-Leu-Ser-Ile- | (SEQ ID NO: 97) |
| Leu-Asp, | |
| Glu-Ile-Glu-Pro-Val-Leu-Ser-Ile- | (SEQ ID NO: 98) |
| Leu-Gln, | |
| Glu-Ile-Glu-Pro-Val-Leu-Ser-Ile- | (SEQ ID NO: 99) |
| Leu-Glu, | |
| Glu-Ile-Glu-Pro-Val-Leu-Ser-Ile- | (SEQ ID NO: 100) |
| Leu-Gly, | |
| Glu-Ile-Glu-Pro-Val-Leu-Ser-Ile- | (SEQ ID NO: 101) |
| Leu-His, | |
| Glu-Ile-Glu-Pro-Val-Leu-Ser-Ile- | (SEQ ID NO: 102) |
| Leu-Met, | |
| Glu-Ile-Glu-Pro-Val-Leu-Ser-Ile- | (SEQ ID NO: 103) |
| Leu-Pro, | |
| Glu-Ile-Glu-Pro-Val-Leu-Ser-Ile- | (SEQ ID NO: 104) |
| Leu-Ser, | |
| Glu-Ile-Glu-Pro-Val-Leu-Ser-Ile- | (SEQ ID NO: 105) |
| Leu-Thr | |
| et | |
| Glu-Ile-Glu-Asp-Val-Leu-Ser-Phe- | (SEQ ID NO: 106) |
| Leu-Gly, | |

these peptides being compounds that can be detected by the procedure defined above, and which present a greater affinity for the PIN than do the other proteins defined above.

These peptides that mimic nNOS are fragments of nNOS, selected by simple or composite mutational analysis and obtained through chemical synthesis.

The two peptides represented by sequences SEQ ID NO: 3 and SEQ ID NO: 4, present one mutation in 9 (Arg mutated to Trp) for the peptide represented by the sequence SEQ ID NO: 3, and three mutations (in 1: Lys mutated to Ile; in 3: Thr mutated to Val; and in 9: Arg mutated to Trp) for the peptide represented by the sequence SEQ IS NO: 4. These two peptides inhibit the interaction between PIN and nNOS with a Kᵢ (IC50) of 4 μmol/L (inhibition constant corresponding to an inhibition percentage of 50%) for the peptide represented by sequence SEQ ID NO: 3, and 0.4 μmol/L for the peptide represented by sequence SEQ ID NO: 4, as shown in Examples 2 and 3.

SEQ ID NO: 3 to SEQ ID NO: 106 correspond to fragments of the mutated nNOS protein.

The invention also concerns nucleic acids that code for one of the proteins, one of the protein fragments, or one of the peptides defined above. It also concerns the nucleotide sequence SEQ ID NO: 1.

SEQ ID NO: 1 is a new nucleic acid sequence that has been identified in the rat. It codes for the new protein corresponding to the pancreatic form of neuronal NOS, represented by the sequence SEQ ID NO: 2.

The invention concerns a pharmaceutical composition characterized in that it contains a protein, a protein fragment or a peptide, as defined above, in association with an acceptable pharmaceutical vehicle.

The dose used can vary from approximately 10 mg to 1 g per day for an adult of average weight of 60 kg.

The invention also concerns a pharmaceutical composition characterized in that it contains all non-peptide substances detected by the screening procedure, as defined above, in association with an acceptable pharmaceutical vehicle.

The intervention also concerns a pharmaceutical composition characterized in that it contains the molecule with the following formula: 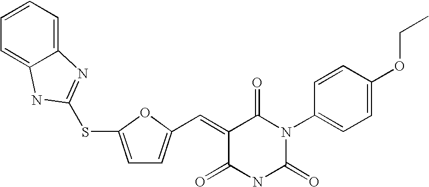
in association with an acceptable pharmaceutical vehicle.

The invention also concerns the use of proteins, protein fragments and peptides, as defined above, for the preparation of drugs for the treatment of prediabetic and hyperinsulinic states, and overt type II diabetes.

The dose used can vary between approximately 10 mg to 1 g per day for an adult of average weight of 60 kg.

The invention also concerns the use of any non-peptide substance detected by the screening procedure, as defined above, for the preparation of drugs for the treatment of prediabetic and hyperinsulinic states, and overt type II diabetes.

The intervention also concerns the use of the molecule with the following formula: 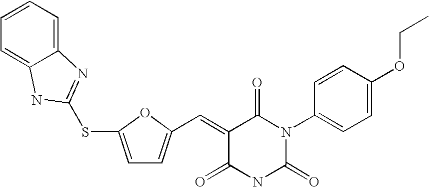
for the preparation of drugs for the treatment of altered insulin response in prediabetic and hyperinsulinic states, and overt type II diabetes.

The prediabetic state is characterized by mild basal hyperglycemia between 6 mM (108 mg/dl) and 7 mM (126 mg/dl). The prediabetic state is also characterized by glucose intolerance, i.e. glycemia between 7.8 mM (140 mg/dl) and 11 mM (200 mg/dl) two hours after an oral glucose test.

Hyperinsulinism corresponds to insulinemia of 1.5 to 10 times the plasma levels given in the literature for a normal man (10 μU/ml 20 pmole/L).

Type II diabetes is characterized by fasting glycemia of greater than or equal to 7 mM (126 mg/dl), and hyperglycemia of over 11 mM (200 mg/dl), 2 hours after an oral glucose test.

The drugs, referred to above and obtained using the proteins, protein fragments, peptides or non-peptide substances, as defined above, are able to re-establish a normal biphasic insulin response in prediabetic and/or hyperinsulinic patients.

“Normal biphasic insulin response” means insulin secretion presenting a first secretion phase of 5-10 minutes, as well as a longer second phase that varies in intensity as a function of glucose intake (approximately 1-2 hours).

The drugs, referred to above and obtained using the proteins, protein fragments, peptides or non-peptide substances, as defined above, are able to re-establish quantitatively normal insulin secretion in type II diabetes patients.

The invention concerns a kit for detecting a modulating compound that reduces complexation between the PIN and nNOS. This kit includes the following:
nNOS, specifically the pancreatic form of nNOS,
PIN,
media or buffers needed for dilution,
materials needed for washing, as necessary
media or buffers needed for the formation of a complex between the PIN and nNOS, and the formation of a complex between the PIN or the nNOS and the compound submitted to the detection procedure,
means to detect variation, specifically a decrease in the quantity of complex formed with the nNOS or with the PIN.

The media or buffers required for the dilution are:
PBS (137 mM NaCl; 2.7 mM KCl; 4.3 mM Na₂HPO₄; 1.4 mM K₂HPO₄),
0.1% Tween 20 in PBS and 1% BSA (bovine serum albumin).

An appropriate washing medium is 0. 1% Tween 20 in PBS.

The media and buffers used for complex formation between the PIN and nNOS, and between the PIN or nNOS and the compound submitted to the detection procedure are 0.1% Tween 20 in PBS and 1% BSA.

The methods for detecting variation in the quantity of complex formed between the nNOS and PIN are as follows:
use of an anti-tag antibody (GST or (HIS)₆) labelled with peroxidase or alkaline phosphatase,
use of an anti-nNOS antibody labelled with peroxidase- or alkaline phosphatase,
use of an anti-tag antibody (GST or (HIS)₆) or a radiolabelled, biotinylated or fluorescent anti-nNOS,
use of two radio- or fluorescent-labelled proteins,
use of surface plasmon resonance with non-labelled nNOS and PIN.

### DESCRIPTION OF FIGURES

FIG. 1 shows the results of RT-PCR analysis of PIN expression in rat pancreatic islets and INS-1 cells (Asfari et al., 1992). The total RNA is isolated, and the complementary DNA synthesized by reverse transcription, then amplified by PCR with primers based on the sequence of the PIN or β₂microglobulin (β₂m), the latter being used as a positive control. A negative control is done in the absence of complementary DNA (C). DNA fragments of known size (2000, 1200, 800, 400, 200 and 100 base pairs) are used as molecular weight (MW) markers.

FIG. 2 shows the results of protein transfer analysis (Western Blot) for the presence of PIN in INS-1 cells. The proteins extracted from the INS-1 cells and from rat brain (Brain) are separated on 13.5% tricine gel, transferred to a nitrocellulose membrane, then incubated with monoclonal anti-PIN antibodies. Detection is done using peroxidase-conjugated anti-mouse antibodies, followed by chemiluminescence analysis. 16 and 7 kDA indicate the molecular weight markers.

FIGS. 3A and 3B show the colocalization of PIN and neuronal NO synthase in INS-1 cells by immunofluorescence. The INS-1 cells are double-labelled with a monoclonal anti-PIN antibody (FIG. 3A), and with rabbit anti-nNOS antibody (FIG. 3B). The fluorescence is detected using a fluorescein-conjugated anti-mouse antibody and a rhodamine-conjugated anti-rabbit antibody, then analyzed using a two-channel confocal microscope. The scale bar represents 10 μm.

FIGS. 4A and 4B show the effect of PIN overexpression in INS-1 cells on glucose-induced insulin secretion.

FIG. 4A shows the results of RT-PCR analysis of PIN overexpression in INS-1 cells. The INS-1 cells are transfected with an expression vector that is either empty (bar C) or that contains PIN complementary DNA (bar PIN). The total RNA is isolated and the complementary DNA amplified by RT-PCR with primers based on the PIN sequence. DNA fragments of known size (2000, 1200, 800, 400, 200 and 100 base pairs) are used as molecular weight markers (bar MW).

FIG. 4B shows the results for the analysis of insulin secretion by INS-1 cells that overexpress PIN (bar PIN) with respect to control cells (bar C). Forty-eight hours after transfection, the cells are incubated in 1 g/L glucose and insulin secretion measured by radioimmunological assay.

FIGS. 5A and 5B present sensorgrams obtained from surface plasmon resonance analysis of the interaction between PIN and a normal nNOS peptide (FIG. 5A), and between PIN and a mutated nNOS peptide (FIG. 5B), the peptide sequence being SEQ ID NO: 3. The peptides are immobilized in a channel of a CM5 biosensor (Biacore AB), and the PIN protein, produced by thrombin digestion of GST-PIN, is injected in increasing concentrations: 5 μg/ml (dashed lines—Curve 4 in FIG. 5A and Curve d in FIG. 5B); 10 μg/ml (dashed-dotted lines—Curve 3 in FIG. 5A and Curve c in FIG. 5B); 20 μg/ml (dotted lines—Curve 2 in FIG. 5A and Curve b in FIG. 5B); and 40 μg/ml (solid lines—Curve 1 in FIG. 5A and Curve a in FIG. 5B). The binding of PIN to the normal peptide (FIG. 5A) and to the mutant peptide (FIG. 5B) are recorded on the sensorgrams, making it possible to determine the association and disassociation constants. These sensorgrams give the response in RU as a function of time, where 1 RU (resonance unit) corresponds to 1 pg of protein per mm² on the biosensor surface.

FIGS. 6A and 6B show the inhibition of the binding of GST-PIN to nNOS for different concentrations of normal and mutant peptides of sequences SEQ ID NO: 3 and SEQ ID NO: 4. After immobilizing the nNOS on an ELISA plate, GST-PIN, previously incubated with increasing concentrations of peptide, was added. The interaction between the two proteins was detected by peroxidase-conjugated anti-GST antibody, and measuring absorbance at 490 nm.

FIG. 6A shows absorbance as a function of peptide concentration (in μg/ml). The curve with dots is for the normal peptide, the curve with squares for peptide SEQ ID NO: 3, and the curve with diamonds for peptide SEQ ID NO: 4.

FIG. 6B shows the percentage of inhibition of the binding of PIN to nNOS as a function of peptide concentration (in μM). The curve with dots is for the normal peptide, the curve with squares for peptide SEQ ID NO: 3, and the curve with diamonds for peptide SEQ ID NO: 4.

FIGS. 7A, 7B, 7C and 7D present sensorgrams for the surface plasmon resonance analysis of the inhibition of binding between the PIN and nNOS by a normal nNOS peptide (FIG. 7A) and by mutated nNOS peptides SEQ ID NO: 3 and SEQ ID NO: 4 (FIGS. 7B and 7C). FIGS. 7A, 7B and 7C give the response in RU, where 1 RU (resonance unit) corresponds to 1 pg of protein per mm² on the biosensor surface.

FIG. 7A shows the inhibition of binding of PIN to nNOS by the normal peptide (Lys Asp Thr Gly Ile Gln Val Arg Asp). Curve 1 corresponds to the absence of peptide; Curves 2, 3 and 4 to concentrations of normal peptides of 20 μm/ml, 50 μg/ml and 100 μg/ml, respectively.

FIG. 7B shows the inhibition of the binding of PIN to nNOS protein by a mutant peptide with sequence SEQ ID NO: 3. Curve a corresponds to the absence of this protein; Curves b, c, d, e and f to peptide concentrations of 5 μg/ml, 10 μg/ml, 20 μg/ml, 30 μg/ml and 40 μg/ml, respectively.

FIG. 7C shows inhibition of the binding of the PIN protein to the nNOS protein by a mutant peptide SEQ ID NO: 4. Curve 1 corresponds to the absence of this protein; Curves 2, 3, 4, 5 and 6 to peptide concentrations of 1 μg/ml, 2 μg/ml, 3 μg/ml, 5 μg/ml and 10 μg/ml, respectively.

FIG. 7D shows the inhibition curve for the binding of PIN to nNOS protein by the mutant peptide SEQ ID NO: 3 (Curve a, dots) and the mutant peptide SEQ ID NO: 4 (Curve b, squares). This figure shows the inhibition percentage for the binding between PIN and nNOS as a function of mutant peptide concentration in μM.

FIG. 8 shows insulin secretion results for pancreatic islets from Zucker (fa/fa) rats in the presence of increasing concentrations of the molecule C₂₄H₁₈N₄O₅S, with respect to insulin secretion obtained in the absence of this molecule. After isolation, the rat islets are stabilized in 0.75 g/L glucose, then incubated in groups of three in 2 g/L glucose with or without this molecule. Insulin secretion is then measured by radioimmunological assay.

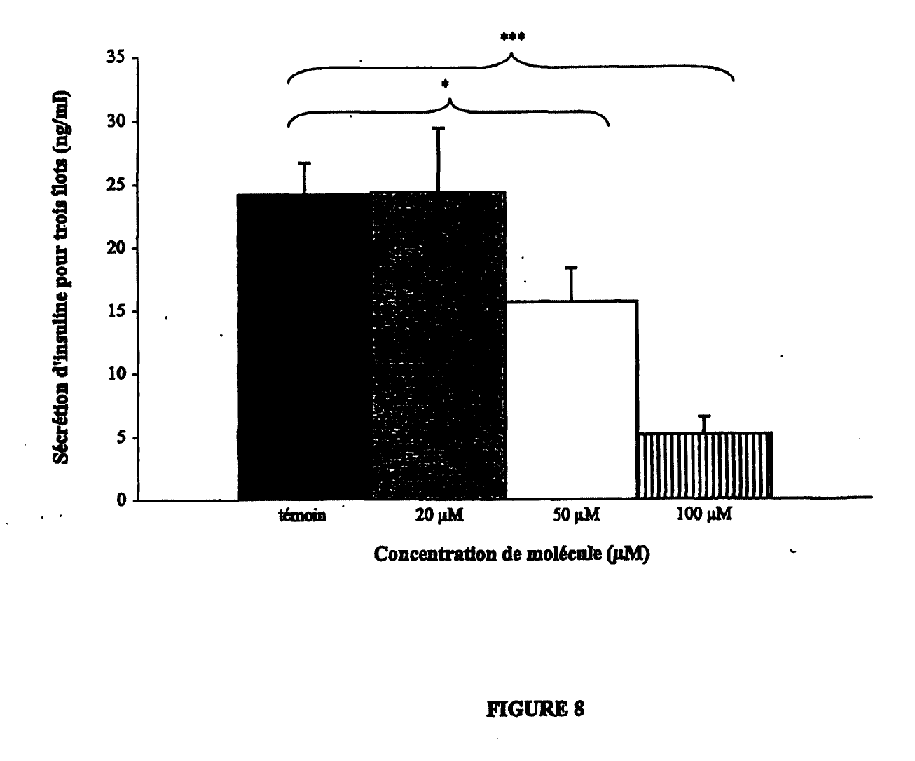

In FIG. 8, insulin secretion (in ng/ml) is represented as a function of the concentration of the said molecule. The black bar shows insulin secretion for the control (without this molecule), the gray bar for the molecule at concentration 20 μM; the white bar for the molecule at concentration 50 μM; and the hatched bar for the molecule at concentration 100 μM. Significance levels are indicated by P<0.05 (*) and P<0.001 (***) with respect to a control (number of replications: n=5).

### MATERIALS AND METHODS

Sequencing the Complementary DNA of the Neuronal Nitric Oxide Synthase Isoform Present in Pancreatic β-cells

Interlocking fragments of complementary DNA (of 450-650 base pairs) are obtained by RT-PCR from rat islets of Langerhans and from the insulin-secreting cell line INS-1 (Asfari et al., 1992). The islets are isolated from the pancreas of male Wistar rats using collagenase digestion, then separated from the exocrine tissue by centrifugation on a Ficoll density gradient (Shibata et al., 1976). The INS-1 cells come from rat insulinoma, and are cultured in RPMI 1640 containing 10% fetal calf serum, 100 U/ml penicillin, 100 μg/ml streptomycin, 2 mM L-glutamine, 10 mM HEPES, 1 mM sodium pyruvate, and 50 μM β-mercaptoethanol. Total RNA from the isolated islets and INS-1 cells are extracted using TRIzol (Life Technologies). The first strand of complementary DNA is synthesized from 10 μg of total RNA in the presence of 3 μg of random primers (Life Technologies), 1 μg of Oligo(dT) primer (Life Technologies), and Superscript II RNase H-Reverse Transcriptase (Life Technologies). The PCR is then done in the presence of Taq Polymerase (Life Technologies), using the primer pairs listed in the table below.

Clockwise and counterclockwise primers used in the
sequencing of the pancreatic form of neuronal NO synthase

| | |
|---|---|
| CLOCKWISE | COUNTERCLOCKWISE |

| | |
|---|---|
| 5′ ATGGAAGAGAACACGTTTGGGGTT 3′ | 5′ TTAGCTTGGGAGACTGAGCCAGCT 3′ |
| 5′ CCAGTCATTAGCAGTAGACAGAGT 3′ | 5′ CATCTTCTGGCTTCCGCGTGTGCT 3′ |
| 5′ TCCTCAAGGTCAAGAACTGGGAGA 3′ | 5′ AGGTCCTTAAACCAGTCGAACTTG 3′ |
| 5′ ATCCAGCCAATGTGCAGTTCACGG 3′ | 5′ GTTCCATGGATCAGGCTGGTATTC 3′ |
| 5′ CCTGTCTTCCACCAGGAGATGCTC 3′ | 5′ TCCCAGTTCCTCCAGGAGGGTGTC 3′ |
| 5′ CCCTGGCCAATGTGAGGTTCTCAG 3′ | 5′ GCATTCACGAGGTCCTCGTGGTTG 3′ |
| 5′ TTCGTGCGTCTCCACACCAACGGG 3′ | 5′ TATTCTGTTGAGCCAGGAGGAGCA 3′ |
| 5′ GGTGCACCTCACTGTGGCCATCGT 3′ | 5′ TTAGGAGCTGAAAACCTCATCTGC 3′ |

After initial 5-minute denaturation at 94° C., the PCR is run for 40 cycles—a denaturation stage at 94° C. for 1 minute, a hybridization stage at 60° C. for 1 minute, an elongation stage at 72° C. for 1 minute, then a final elongation of 10 minutes.

After migration on 1.5% agarose gel, the complementary DNA fragments are purified using the QIAEX II Gel Extraction Kit (Qiagen). The fragments are then manually sequenced twice using dCTP [αS³⁵] and the Thermo Sequenase Cycle Sequencing Kit (Amersham), and once using an automatic sequencer (ABI PRISM 377, PE Applied Biosystems) and the dRhodamine Terminator Cycle Sequencing Ready Reaction Kit (PE Applied Biosystems). As can be seen from sequence SEQ ID NO: 2, four point mutations are identified in the sequence of the pancreatic form of neuronal NO synthase, which means it is 99.8% homologous to the sequence for the rat brain form. Three of these mutations modify the amino acid sequence: One valine is mutated to isoleucine in position 269, one alanine to praline in position 953, and one serine to phenylalanine in position 1008. Pancreatic NO synthase is therefore slightly different from the neuronal NO synthase previously identified in rat brain. (Bredt et al., 1991).

Presence of PIN Messenger RNA in Endocrine Pancreatic β-cells

To show the presence of messenger RNA in the protein inhibitor of neuronal nitric oxide synthase (PIN) (Jaffrey et al., 1996) in endocrine pancreatic β-cells, rat islets of Langerhans and the insulin-secreting cell line INS-1 are used as sources of pancreatic β-cells. The total RNA of the isolated islets and the INS-1 cells are extracted with TRIzol (Life Technologies). The first strand of complementary DNA is synthesized from 10 μg total DNA in the presence of 3 μg random primers (Life Technologies), 1 μg of oligo(dT) primer (Life Technologies) and Superscript II RNase H-reverse transcriptase (Life Technologies). The PCR is then run using Taq Polymerase (Life Technologies) with the following primer pairs:

for PIN:
_{5′}TTGAGCGGCGCCAGCACCTTCCCT_{3′}
and
_{5′}CGAGGTGTTCCCTTAGCAAGGCTG_{3′}
for the β2-microglobulin:
_{5′}ATCTTTCTGGTGCTTGTCTC_{3′}
and
_{5′}AGTGTGAGCCAGGATGTAG_{3′}

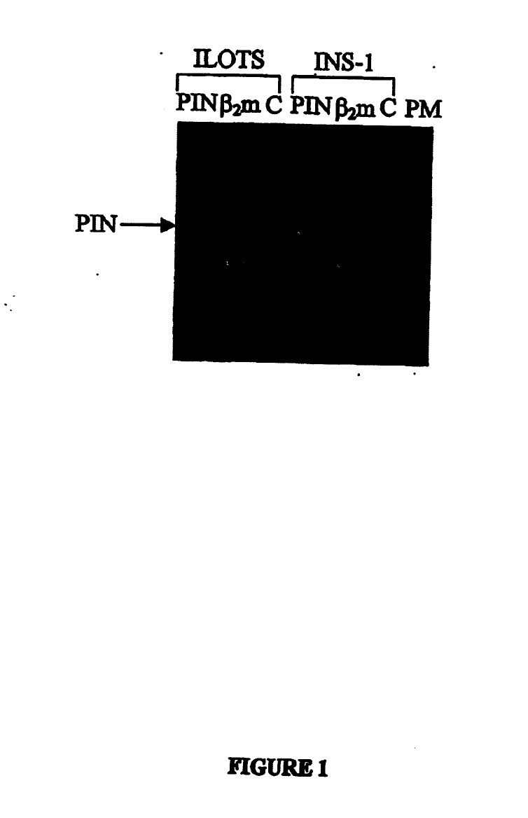

After initial 5-minute denaturation at 94° C., the PCR is run for 40 cycles—a denaturation stage at 94° C. for 1 minute, a hybridization stage at 60° C. for 1 minute, an elongation stage at 72° C. for 1 minute, then a final elongation of 10 minutes. The PCR products are then separated on 1.5% agarose gel and visualized by ethidium bromide staining. A fragment of the expected size (443 base pairs) is obtained with the PIN primers in both the pancreatic islets and the INS-1 cells (see FIG. 1). The RT-PCR analysis, therefore, reveals the presence of messenger RNA from PIN in rat pancreatic β-cells. Accordingly, the simultaneous expression of neuronal NO synthase and its natural inhibitor PIN in the insulin-secreting cells of the endocrine pancreas is demonstrated. The PIN complementary DNA is sequenced, and its complete homology with the sequence of rat brain PIN verified (Faffrey et al., 1996).

Presence of PIN in INS-1 Cells

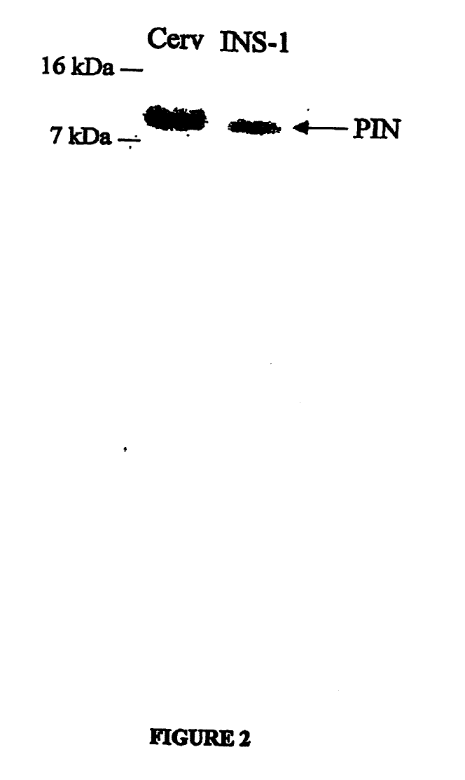

The INS-1 cells and rat brains are homogenized in lysis buffer containing 50 mM Tris (pH 7.4), 150 mM NaCl, 2 mmol/L EDTA, 1 mmol/L phenylmethylsulfonyl fluoride, 10 μg/ml leupeptin and 10 μg/ml aprotinin. The insoluble material is eliminated by centrifugation. The concentration of proteins in the supernatant is determined by Coomassie Blue staining (Coomassie Protein Assay Reagent, Pierce). Then 80 μg of proteins are separated by electrophoresis on 13.5% tricine gel, and transferred to a nitrocellulose membrane. The membranes are first saturated in 5% skim milk powder in 0.1% Tween 20 in PBS, then incubated overnight with a monoclonal anti-PIN antibody (diluted 1:250, Transduction Laboratories). After three washings in PBS-Tween, the membrane is incubated with a peroxidase-conjugated anti-mouse antibody (diluted 1:5,000, Sigma Aldrich). Immunoreactivity is detected by chemiluminescent assay (ECL, Amersham Life Science). A Western Blot reveals that the PIN is identical in size to the PIN expressed in rat brain (see FIG. 2).

Colocalization of PIN with Neuronal NO Synthase in INS-1 Cells

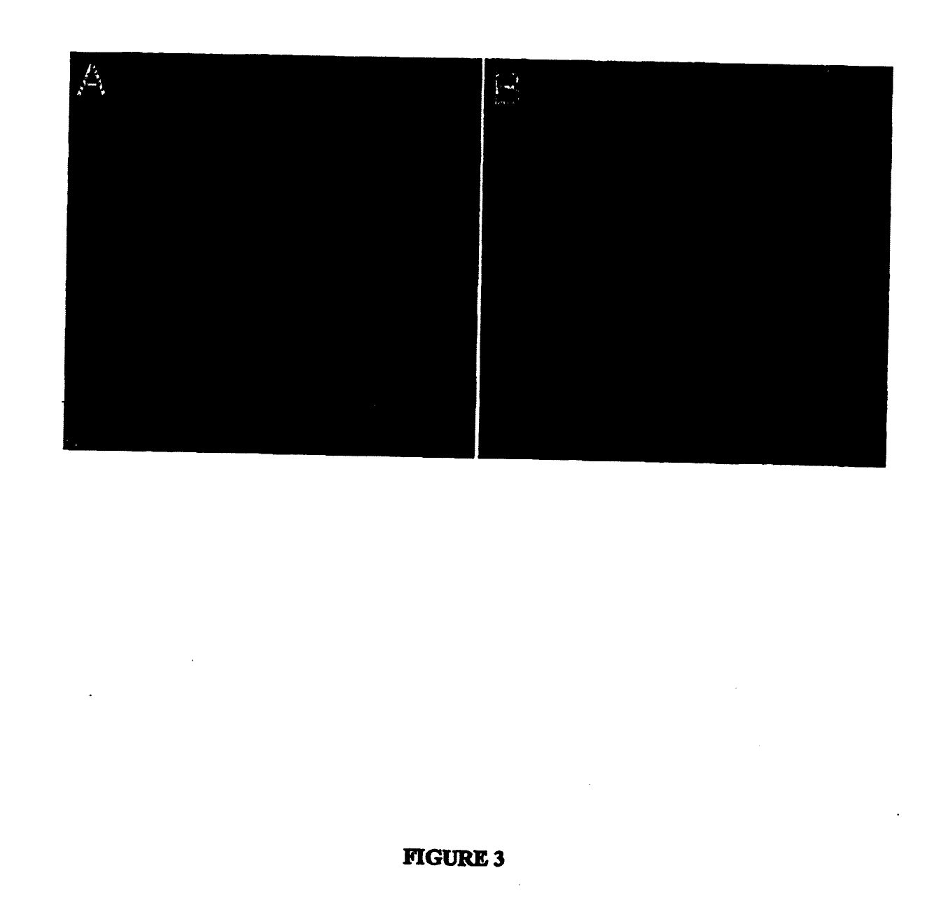

The INS-1 cells are seeded in Lab-Tek® Chamber Slide Systems and cultured for 4 days before use. They are then fixed with 2% paraformaldehyde in PBS (phosphate buffer saline) for 20 minutes and permeabilized for 5 minutes with 0.1% Triton X-100. After saturation of the non-specific sites with 2% BSA (beef serum albumin), the cells are incubated overnight with a monoclonal anti-PIN antibody (diluted 1:100, Transduction Laboratories) and a rat neuronal anti-NO synthase antibody (diluted 1:100, Euro-Diagnostica). After several washings, a fluorescein-conjugated anti-mouse antibody (diluted 1:100, Biosys) and a rhodamine-conjugated anti-rabbit antibody (diluted 1:100, Biosys) are applied to the cells for 1 hour. After several washings, the cells are placed in Citifluor (Citifluor Ltd.) and observed under a confocal microscope using argon and krypton lasers (Biorad). PIN is present in the cytoplasm of INS-1 cells (FIG. 3A). In addition, the fluorescent signals of PIN (FIG. 3A) and neuronal NO synthase (see FIG. 3B) are very similar, indicating that the two proteins are strongly colocalized in rat pancreatic β-cells. In effect, it has been shown that PIN interacts with neuronal NO synthase in vitro and in vivo (Jaffrey et al., 1996) at amino acids 163-245. It therefore seems that neuronal NO synthase and PIN interact inside pancreatic β-cells.

Modulating Role of PIN on Glucose-induced Insulin Secretion in the INS-1 Cell Line

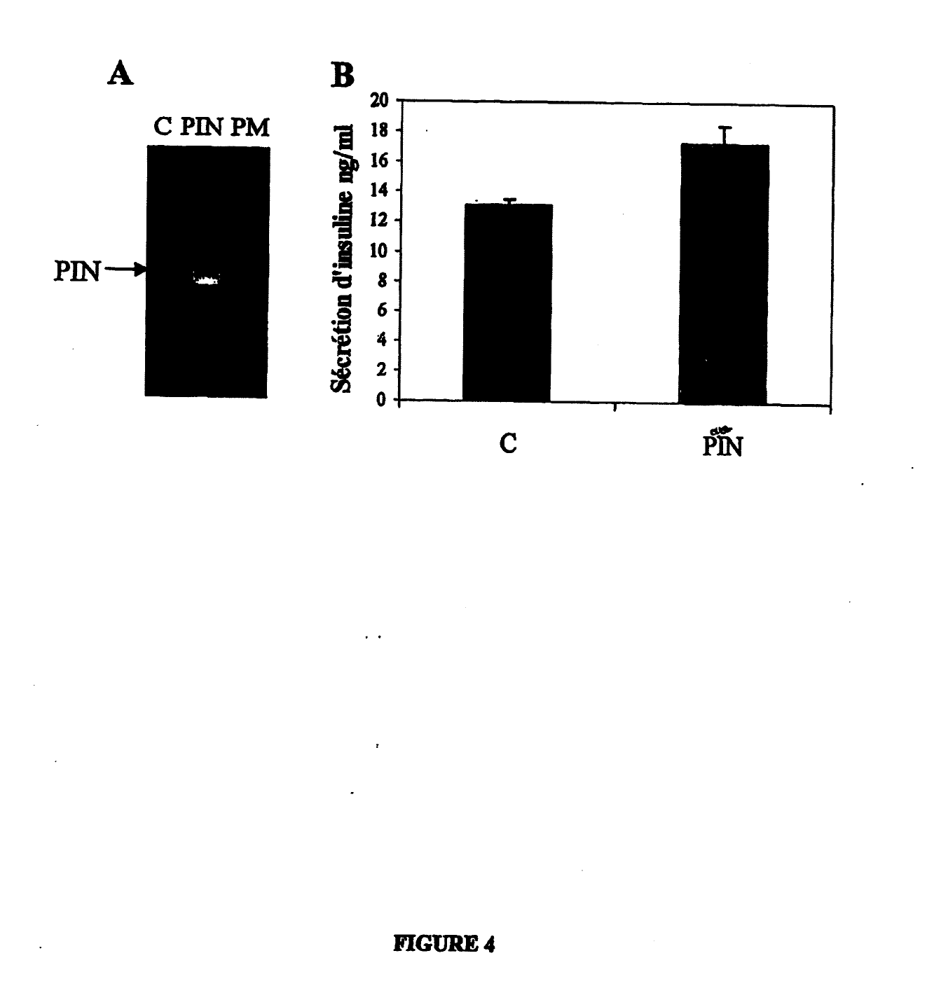

PIN is overexpressed in INS-1 cells and the insulin response to glucose measured. PIN complementary DNA obtained by RT-PCR (see above) is cloned in the eukaryotic expression vector pCR3.1 (TA Cloning Kit, Invitrogen). The INS-1 cells (approximately 8.10⁵) are then transfected with 1.5 μg plasmid (empty or containing PIN) using LipofectAMINE PLUS Reagent (Life Technologies). The overexpression of PIN is then verified by RT-PCR using 5 μg total RNA and the primers listed above (see Table 1). Then, 48 hours after transfection, the cells are washed in Krebs-Ringer bicarbonate buffer (pH 7.4) (108 mM NaCl; 1.19 mM KH₂PO₄; 4.74 mM KCl; 2.54 mM CaCl₂; 1.19 mM MgSO₄, 7H₂O; 18 mM NaHCO₃) without glucose, then preincubated in the same buffer for 1 hour at 37° C. After discarding the buffer, the cells are incubated in Krebs containing 1 g/L glucose for 1 hour at 37° C. The supernatant is then recovered and insulin secretion measured by radioimmunological assay (Herbert et al., 1965). Approximately 5 times as much PIN messenger RNA is present in cells after transfection of the PIN vector with respect to the control cells (FIG. 4A). In addition, glucose-induced insulin secretion increases 25% in cells overexpressing with PIN with respect to control cells (FIG. 4B). It appears, therefore, that PIN plays a positive modulating role in glucose-induced insulin secretion.

Screening Test Procedure

To produce the PIN, PIN complementary DNA obtained by RT-PCR (see Example 1 below) is cloned into the vector pET21b (containing a polyhistidine tag in the C-terminal position, (HIS)₆ (e.g. Novagen) and the vector pGEX-2T (containing a glutathion s-transferase tag in the N-terminal position, and GST (e.g. Pharmacia). After transformation of BL21 bacteria (DE3) (e.g. Novagen) by recombinant plasmids, the bacteria are cultured at 37° C. in LB buffer to an OD of 0.6. The protein is then produced by 1 mM IPTG (isopropylthio-β-D-galactoside) induction for 5 hours at 30° C. The bacteria are recovered by centrifugation and then lyzed under standard conditions (Short Protocols in Molecular Biology, 2nd Edition, John Wiley and Sons). The insoluble material is eliminated by centrifugation and the protein is purified on a nickel column for the polyhistidine tag (e.g. Ni NTA agarose, Qiagen), or a glutathione sepharose column, for the GST tag (e.g. Pharmacia), as per the manufacturer's recommendations. The PIN is then stored at −80° C.

To obtain the pancreatic nNOS, the polyhistidine-tagged complementary DNA of the pancreatic nNOS is cloned into the vector p119L (Poul et al., 1995) under the control of viral promoter P10 (Poul et al., 1995). The recombinant virus is obtained by cotransfection of the loaded vector and baculovirus DNA in Sf9 insect cells (ATCC CRL 1711) using lipofection (DOTAP, Roche Diagnostics). The virus clones are then isolated using the lysis plaque method and selected for their ability to produce nNOS by protein transfer (Western Blot) with an anti-nNOS antibody (Transduction Laboratories). The protein is purified on a nickel column (e.g. Ni NTA agarose, Qiagen) as per the manufacturer's recommendations. The pancreatic nNOS is then stored at −80° C.

In the first the method described in the invention, the pancreatic nNOS is immobilized overnight at the bottom of a plastic MaxiSorp plate (Nunc) at a concentration of 1-5 μg/ml in 200 μl PBS at 4° C. After washing in PBS containing 0.1% Tween 20, the plate is saturated with 100 μl PBS/1% BSA for 1 hour at 37° C., then incubated with the GST (GST-PIN)- or polyhistidine (PIN-(HIS)₆)-tagged PIN at a concentration of 0.1-10 μg/ml in 100 μl of PBS/0.1% Tween 20/1% BSA in the presence or absence of the test compound for 2 hours at 37° C. The plate is washed and then incubated with 100 μl of anti-tag antibodies, anti-GST or peroxidase-conjugated anti-(HIS)₆ (diluted 1:2000 in PBS/0.1% Tween 20/1% BSA, Sigma Aldrich) for 1 hour at 37° C. The formation of the nNOS-PIN-antibody complex is detected by a colour reaction in the presence of the peroxidase substrate, O-phenylenediamine, for 30 minutes in the dark. The intensity of the colour is measured at 490 nm.

In the second method described in the invention, the PIN, in the form of GST-PIN or PIN-(HIS)₆, is immobilized at the bottom of a plastic plate, then placed in contact with the pancreatic nNOS. Reactivity is then detected using a peroxidase-conjugated anti-nNOS antibody.

### EXAMPLE 1

This example shows that a mutant peptide of the nNOS, i.e. that represented by sequence SEQ ID NO: 3, presents better affinity for PIN than does the same non-mutant peptide of the nNOS (Lys Asp Thr Gly Ile Gln Val Asp Arg Asp), according to surface plasmon resonance analysis.

The soluble peptides of the nNOS are prepared by a AMS 422 robot (Abimed) using Fmoc solid phase peptide synthesis (Gausephol, 1992). These nNOS peptides containing 10 amino acids correspond to the region of interaction with PIN (nNOS amino acids 229-238). The peptides are deprotected and their resin removed by treatment with trifluoroacetic acid using appropriate sensors. The peptides are lyophilized and their purity verified by analytic HPLC. If necessary, the peptides are then purified to 90% by preparative HPLC, then analyzed using mass spectrometry.

The binding of the PIN to the immobilized peptides is analyzed using BIAcore 2000 (Biacore AB). The peptides, at a concentration of 10 μg/ml in 10 mM (pH 4) acetate buffer, are conjugated on a channel of a CM5 biosensor (Biacore AB), using the NHS/EDC protocol (N-hydroxysuccinimide (NHS), Biacore AB; N-ethyl-N′-(dimethylaminopropyl) carbodiimide (EDC), Biacore AB), which produces a density of immobilized peptides of approximately 100 pg/mm². Increasing concentrations of PIN (5, 10, 20 and 40 μg/ml), produced by thrombin digestion of GST-PIN (Pharmacia), are injected on the biosensor at flux 30 μl/min, and sensorgrams generated for the binding of PIN-peptide binding (association time of 180 seconds; dissociation time of 400 seconds). The association and dissociation constants are determined from the sensorgram using BIAevaluation 3.0 software (Biacore AB) and a global type of analysis (simultaneous analysis of kinetic association and dissociation constants for sensorgrams at all peptide concentrations used). As a control, the appropriate peptide (Lys Ala Val Asp Leu Ser His Gln Pro Ser Ala Ser Lys Asp Gln Ser Leu), which is a fragment of the nNOS (bounded by the amino acids at positions 131 and 147 of the nNOS protein, see FIG. 1) but does not correspond to the interaction region between the PIN and nNOS proteins, is immobilized in the same way and added to the PIN at the same concentrations.

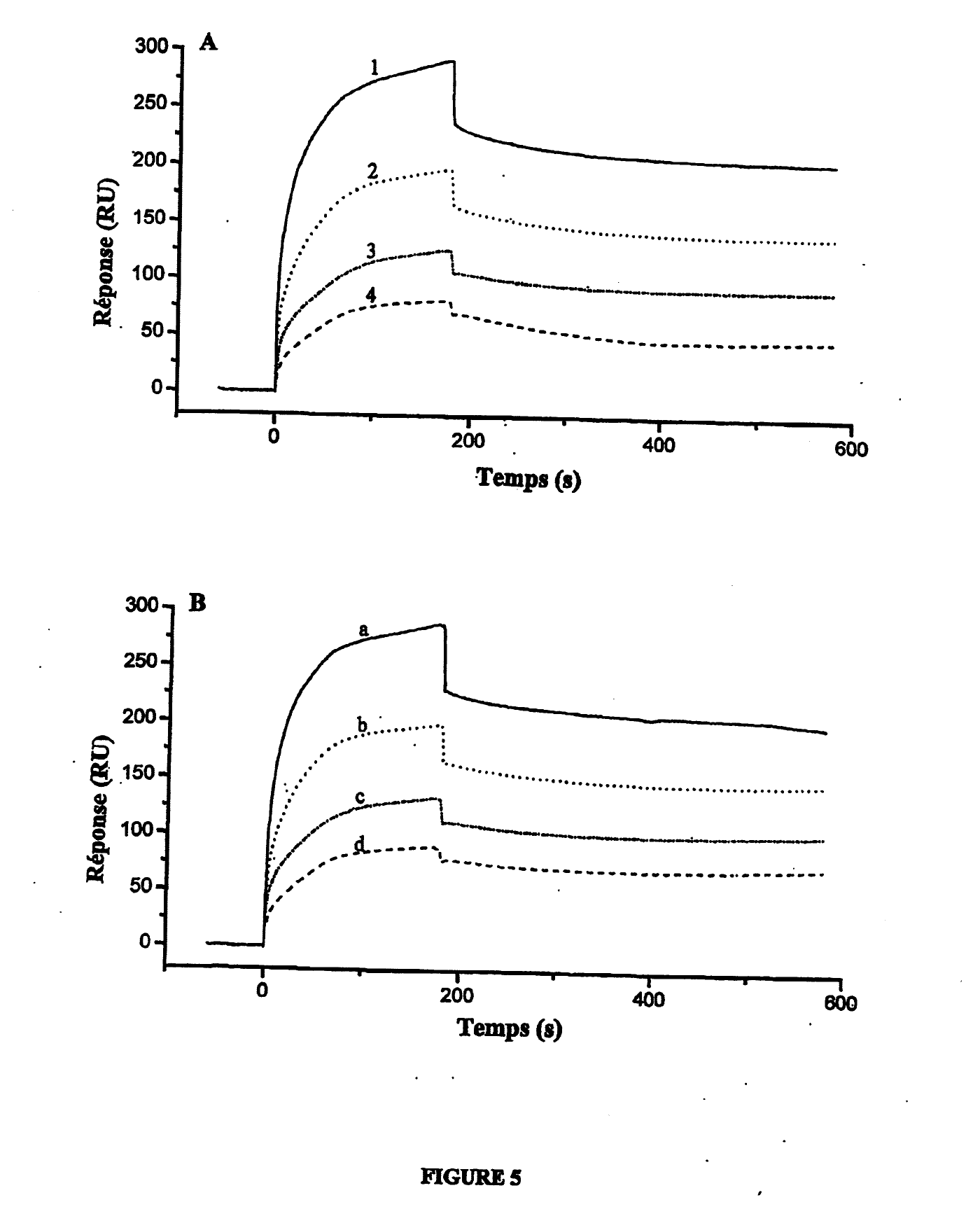

The affinity of the PIN protein is two times greater for the mutant peptide represented by the sequence SEQ ID NO: 3 (see FIG. 5B, Kₐ=1.86×10⁸; K_{d}=5.38×10⁻⁹) than for the normal peptide (see FIG. 5A, Kₐ=8.43×10⁷; K_{d}=1.19×10⁻⁸). Introducing a mutation in the nNOS peptide (SEQ ID NO: 3, arginine mutated to tryptophane in position 9) increases its affinity for the PIN protein.

### EXAMPLE 2

This example uses the first method described in the invention (see above) to show that mutant peptides, represented by sequences SEQ ID NO: 3 and SEQ ID NO: 4, that present better affinity for PIN than nNOS, are able to reduce the attraction between nNOS and PIN.

The nNOS protein is immobilized on a ELISA MaxiSorp plate (Nunc) at a concentration of 1 μg/ml in 100 μl PBS overnight at 4° C. After washing in PBS containing 0.1% Tween 20, the plate is saturated with 100 μl of PBS/1% BSA for 1 hour at 37° C. The 0.5 μg/ml GST-PIN in 100 μl of PBS/0.1% Tween 20/1% BSA, is first incubated with the given peptide (as described above), the normal peptide (Lys Asp Thr Gly Ile Gln Val Asp Arg Asp) or the mutant peptides (SEQ ID NO: 3 or SEQ ID NO: 4) at increasing concentrations (0.010-100 μg/ml for mutant peptides; 10-1,000 μg/ml for non-mutant peptides) in the same buffer for 1 hour at 37° C. After incubating the peptide-PIN mixture for 2 hours on the plate, the plate is washed, then incubated with 100 μl of peroxidase-conjugated anti-GST antibody (diluted 1:2000 in PBS/0.1% Tween 20/1% BSA, Sigma Aldrich) for 1 hour at 37° C. Complex formation between the PIN and nNos is detected by adding the mixture to O-phenylenediamine, a peroxidase substrate, for 20 minutes in the dark, then measuring the intensity of colouration at 490 nm. A control is done for each peptide, as described above.

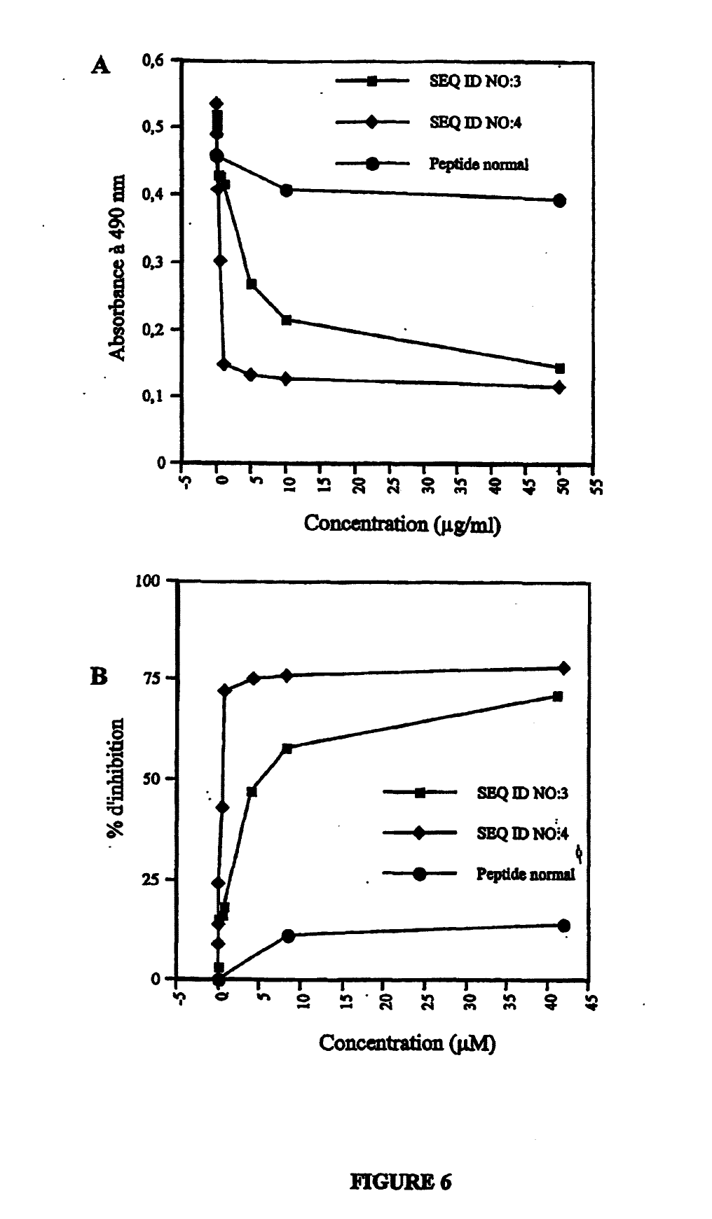

In the presence of the normal nNOS peptide (see FIG. 6A), the binding of the PIN to the nNOS is inhibited by 14% for a concentration of the said peptide of 50 μg/ml. By contrast, the two mutant peptides represented by sequences SEQ ID NO: 3 and SEQ ID NO: 4 cause an inhibition in the binding of the PIN to the nNOS of up to 71% and 78%, respectively, at a concentration of 50 μg/ml.

According to the inhibition curves of the PIN-nNOS binding (see FIG. 6B), the peptide represented by the sequence SEQ ID NO: 3 shows an inhibition constant (Kᵢ:IC₅₀) of 5 μM, the peptide represented by the sequence SEQ ID NO: 4 a constant of 0.5 μM, whereas the normal peptide has an inhibition constant of only 300 μM.

### EXAMPLE 3

This example uses surface plasmon resonance analysis to show that the mutant peptides of the nNOS, represented by sequences SEQ ID NO: 3 or SEQ ID NO: 4, that present better affinity for the PIN protein than the nNOS protein, are able to inhibit the interaction between nNOS and PIN.

The binding of PIN to the immobilized nNOS protein (Alexis) is analyzed using Biacore 2000 (Biacore AB) in the presence or absence of synthetic peptides. The nNOS, at a concentration of 10 μg/ml in 10 mM of acetate tampon (pH 5.5), is conjugated on a channel of a CM5 biosensor (e.g. Biacore AB) using the NHS/EDC protocol, which leads to a density of immobilized protein of approximately 6000 pg/mm². The PIN protein at concentration 5 μg/ml is preincubated in the presence of increasing concentrations of peptides (1-100 μg/ml) for 30 minutes, then injected on the chip at flux 30 μl/min. The sensorgrams showing the binding of the peptides to the nNOS are recorded for an association time of 180 seconds and a dissociation time of 400 seconds. The association and dissociation constants are determined from the sensorgram using BIAevaluation 3.0 software (Biacore AB), and a global analysis method (simultaneous analysis of kinetic association and dissociation constants for sensorgrams at all peptide concentrations used). The controls are done by injecting the given peptide, preincubated with PIN, on the nNOS (as described above).

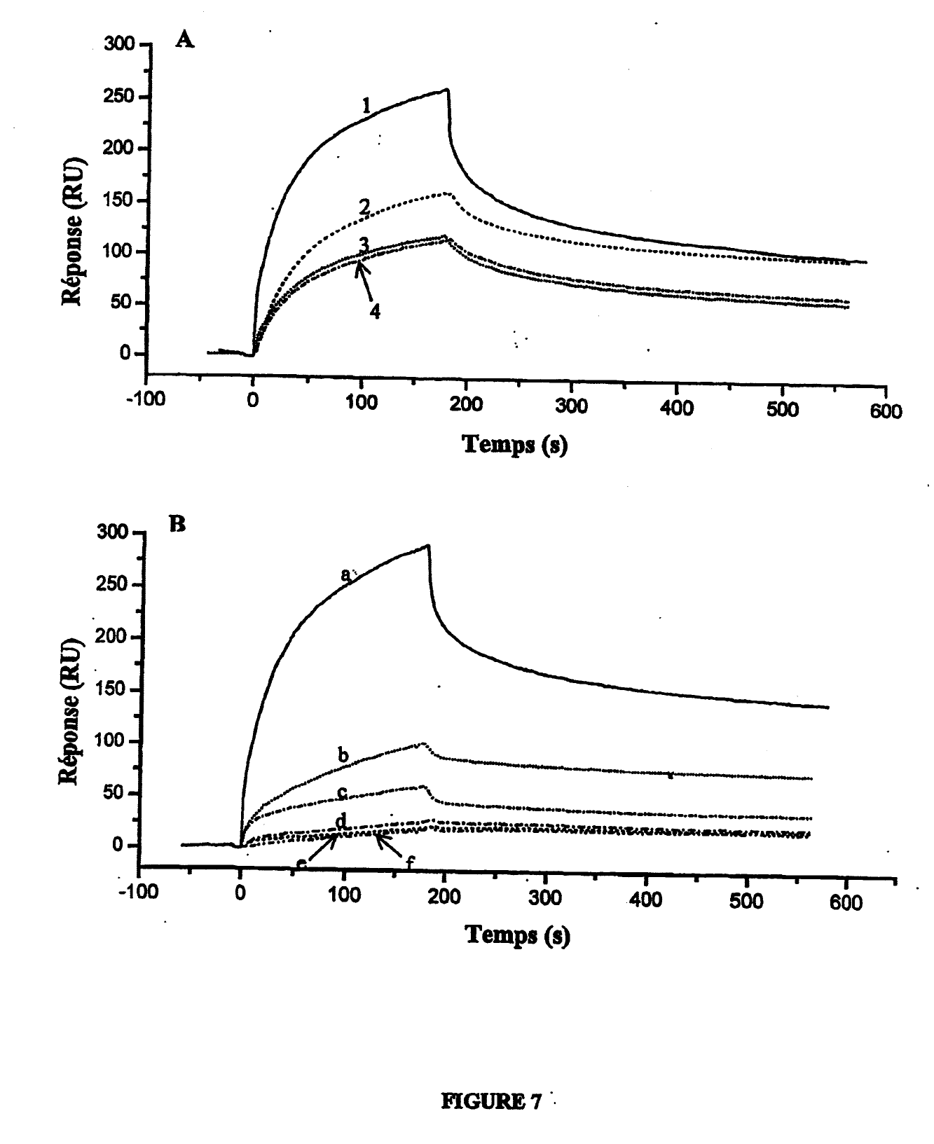

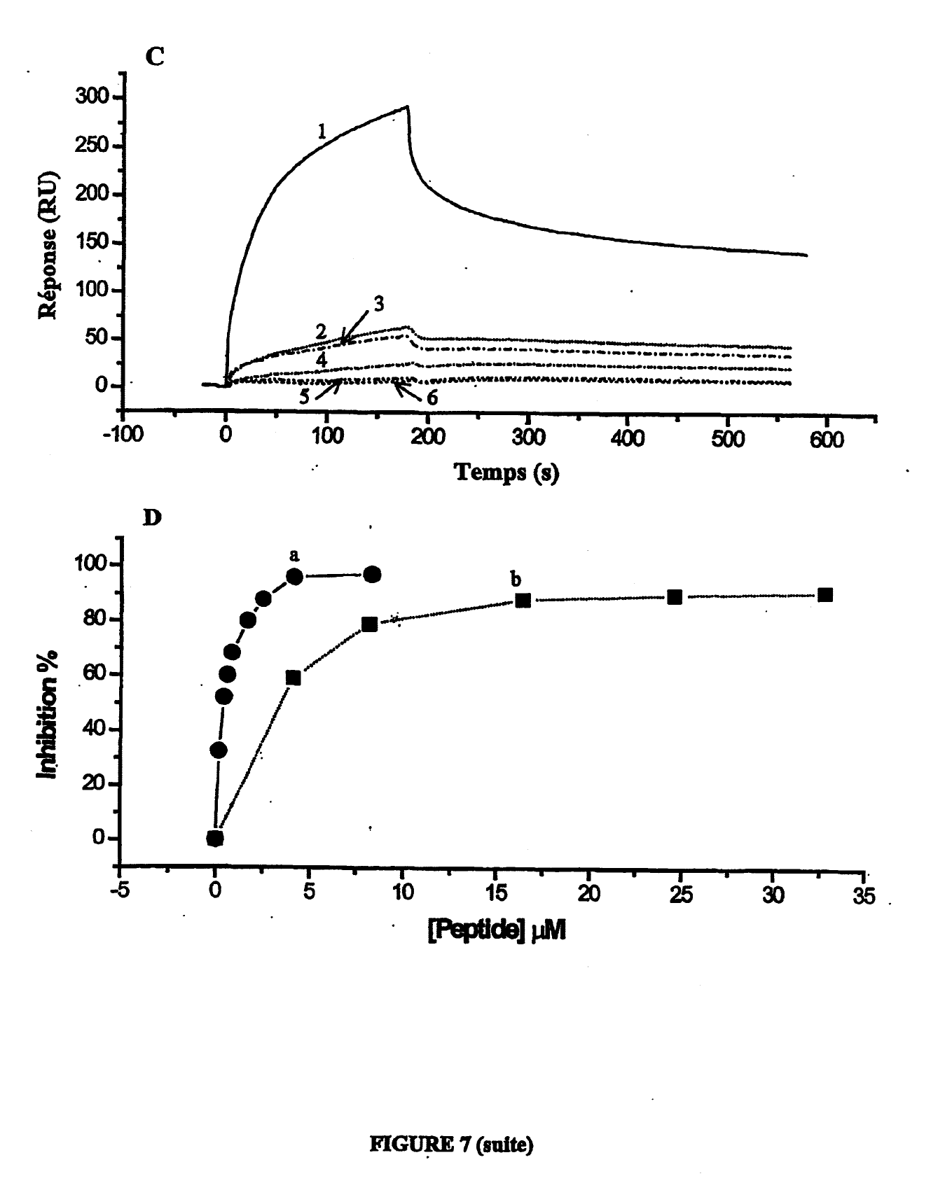

In the presence of the normal nNOS peptide (see FIG. 7A), the binding of the PIN to the nNOS protein is inhibited by 19% at 20 μg/ml of peptide, with inhibition plateauing at 45% at 50 μg/ml and 100 μg/ml of peptide. By contrast, the mutant peptide, represented by the sequence SEQ ID NO: 3 (see FIG. 7B) inhibits the binding of the PIN to the nNOS by 59% at 5 μg/ml of peptide, and blocks this interaction almost completely at peptide concentrations of 30 μg/ml and 40 μg/ml (90% and 91%, respectively). The same is true for the mutant peptide represented by the sequence SEQ ID NO: 4 (see FIG. 7C), which inhibits the binding of PIN to the nNOS by 75% at 1 μg/ml of peptide and completely inhibits it at 10 μg/ml (98%). According to the inhibition curves for the PIN-nNOS binding as a function of the peptide concentration used (FIG. 7D), the peptide represented by the sequence SEQ ID NO: 3 shows an inhibition constant (Kᵢ: IC50) of 4 μM, and the peptide represented by sequence SEQ ID NO: 4 a constant of 0.4 μM. Therefore, the two mutant peptides are able to inhibit the interaction between the PIN and nNOS.

### EXAMPLE 4

This example shows that the molecule C₂₄H₁₈N₄O₅S, obtained by in vitro screening of a bank of 3,000 compounds (Chembridge) as per the invention procedure described above, is able to decrease insulin secretion in hyperinsulinic and insulin-resistant obese animals (Zucker (fa/fa) rats—a line of rats with a mutation in the fa gene (short for “fatty”)).

The chemical molecule used has the following formula:

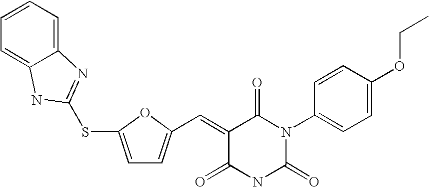

When placed in 100 μl PBS overnight at 4° C., the recombinant nNOS (100 ng), obtained as per the procedure described above, is adsorbed at the bottom of a microplate at a concentration of 1 μg/ml. After saturation in 200 μl of 1% PBS/BSA for 1 hour at 37° C., the nNOS is combined with 5 μl of the molecule (at a final concentration of 10 μM) and 0.5 μg/ml GST-PIN for 2 hours at 37° C. The formation of the PIN-nNOS complex in then detected by incubation with anti-GST antibodies (diluted 1:2000) for 1 hour at 37° C., then detected by incubating with O-phenylenediamine for 30 minutes and measuring absorbance at 490 nm. Molecules are considered positive when they inhibit the interaction by 30-50%.

The islets of Langerhans of Zucker (fa/fa) rats are isolated using the collagenase digestion technique of Lacy et al. (Diabetes, 1967). After isolation, the islets are stabilized in Krebs-Ringer containing 0.75 g/L glucose for 45 minutes at 37° C. Groups of three islets are then incubated in Krebs-Ringer with 2 g/L glucose containing increasing concentrations of the molecule C₂₄H₁₈N₄O₅S (20-100 μM) for 1 hour at 37° C. The liquid supernatant is then recovered and insulin secretion measured by radioimmunology (see FIG. 8).

The molecule C₂₄H₁₈N₄O₅S, in decreasing the PIN-nNOS interaction, blocks in a dose-dependant fashion (starting at a concentration of 50 μM) insulin secretion induced by 2 g/L glucose in islets isolated from hyperinsulinic rats. In effect, for concentrations of 50 and 100 μM, the insulin response is reduced by 36% and 79%, respectively.

Therefore, this molecule is capable of reducing insulin hypersecretion in these prediabetic animals.

### REFERENCES

Asfari et al. (1992) Endocrinology, 130, 167-178
Bredt et al. (1991) Nature, 351, 714-718
Gausephol (1992) Peptide Research, 5, 315-320
Herbert et al. (1965) J. Clin. Endocrinol. Metabol. 25, 1375-1384
Jaffrey et al. (1996) Science, 274, 774-776
Lacy et al. (1967) Diabetes, 16(1), 35-39
Maechler et al. (1997) Embo J., 16(13), 3833-3841
Poul et al. (1995) Immunotechnology, 1, 189-196
Shibata et al. (1976) Diabetes, 8, 667-672
Short Protocols in Molecular Biology, 2nd Edition, John Wiley and Sons

1-14. cancel.

15. A method of detecting compounds that modulate the complexation between neuronal nitric oxide synthase protein (nNOS), represented by the sequence SEQ ID NO:2, or one of its variants and the protein inhibitor of neuronal nitric oxide synthase (PIN), the modulation of this complexation causing a modification of the insulin response regulated by nNOS or one of its variants, in which:
a mixture of the said compound, the PIN and the nNOS or one of its variants is incubated in conditions that enable the:
formation of a complex between the PIN and nNOS or one of its variants,
formation of a complex between the said compound and the PIN, or between the said compound and nNOS or one of its variants;
any significant variation detected in the quantity of complex formed between the PIN and nNOS or one of its variants with respect to a control value corresponds to:
the quantity of complex formed between the PIN and nNOS or one of its variants in the absence of the test compound, or
the absence of a complex between the PIN and nNOS or one of its variants, resulting in the absence of PIN or the absence of nNOS or one of its variants, or
the quantity of complex formed between the PIN and nNOS or one of its variants in the presence of a reference inhibitor; and
when there is significant variation as defined above, it is concluded that there was binding between the said compound and the PIN or between the said compound and the nNOS or one of its variants, leading to modulation of the complexation between the PIN and nNOS or one of its variants.

16. The method of claim 15, wherein the compound does not substantially modify the catalytic activity of the nNOS or one of its variants.

17. A method for detecting compounds that decrease the complexation between neuronal nitric oxide synthase (nNOS) or one of its variants, and the protein inhibitor of neuronal nitric oxide synthase (PIN), the decrease in this complexation leading to a reduction in the insulin response regulated by the nNOS or one of its variants, in which:
a mixture of the said compound, the PIN and the nNOS or one of its variants is incubated in conditions that enable the:
formation of a complex between the PIN and nNOS or one of its variants,
formation of a complex between the said compound and the PIN, or between the said compound and the nNOS or one of its variants;
any significant decrease detected in the quantity of complex formed between the PIN and nNOS or one of its variants with respect to a control value corresponds to:
the quantity of complex formed between the PIN and nNOS or one of its variants in the absence of the compound submitted to the detection procedure, or
the absence of complex formed between the PIN and nNOS or one of its variants, resulting in the absence of PIN or the absence of nNOS or one of its variants, or
the quantity of complex formed between the PIN and nNOS or one of its variants in the presence of a reference inhibitor; and
when there is significant decrease as defined above, it is concluded that there was binding between the said compound and the PIN, or between the said compound and the nNOS or one of its variants, leading to reduction of the complexation between the PIN and nNOS or one of its variants.

18. The method of claim 15 or claim 17, in which variation is detected, specifically any significant decrease in the quantity of complex formed between the PIN and nNOS, with respect to a first, second, and third control value, one of these control values corresponding to the quantity of complex formed between the PIN and nNOS in the absence of the compound submitted to the detection procedure; another to the absence of complex between the PIN and nNOS, resulting in either the absence of PIN or the absence of nNOS; and another to the quantity of complex formed between the PIN and nNOS in the presence of a reference inhibitor.

19. The method of claim 15 or claim 17, in which the mixture of the PIN, nNOS, and the compound submitted to the detection procedure is prepared by:
simultaneously adding the PIN, nNOS, and the compound submitted to the detection procedure, or
consecutively adding the PIN, the compound submitted to the detection procedure, and the nNOS, or consecutively adding the nNOS, the compound submitted to the detection procedure, and the PIN, or
adding the compound previously incubated with the PIN or the nNOS, to the nNOS protein or the PIN, respectively.

20. The method of claim 15 or claim 17, in which the nNOS protein is first fixed on a solid substrate.

21. The method of claim 15 or claim 17, in which the PIN is first fixed on a solid substrate.

22. The method of claim 15 or claim 17, in which the PIN and nNOS are in solution.

23. A protein characterized in that it contains or is constituted by the sequence of SEQ ID NO:2, or fragment of said protein comprising at least 100 amino acids, on condition the said fragment contains the amino acid in position (269).

24. A peptide having the sequence:

| | |
|---|---|
| Lys-Asp-Thr-Gly-Ile-Gln-Val-Asp- | (SEQ ID NO: 3) |
| Trp-Asp, | |
| Ile-Asp-Val-Gly-Ile-Gln-Val-Asp- | (SEQ ID NO: 4) |
| Trp-Asp, | |
| Cys-Asp-Thr-Gly-Ile-Gln-Val-Asp- | (SEQ ID NO: 5) |
| Arg-Asp, | |
| Ile-Asp-Thr-Gly-Ile-Gln-Val-Asp- | (SEQ ID NO: 6) |
| Arg-Asp, | |
| Val-Asp-Thr-Gly-Ile-Gln-Val-Asp- | (SEQ ID NO: 7) |
| Arg-Asp, | |
| Lys-Asp-Ala-Gly-Ile-Gln-Val-Asp- | (SEQ ID NO: 8) |
| Arg-Asp, | |
| Lys-Asp-Cys-Gly-Ile-Gln-Val-Asp- | (SEQ ID NO: 9) |
| Arg-Asp, | |
| Lys-Asp-Glu-Gly-Ile-Gln-Val-Asp- | (SEQ ID NO: 10) |
| Arg-Asp, | |
| Lys-Asp-Ile-Gly-Ile-Gln-Val-Asp- | (SEQ ID NO: 11) |
| Arg-Asp, | |
| Lys-Asp-Lys-Gly-Ile-Gln-Val-Asp- | (SEQ ID NO: 12) |
| Arg-Asp, | |
| Lys-Asp-Phe-Gly-Ile-Gln-Val-Asp- | (SEQ ID NO: 13) |
| Arg-Asp, | |
| Lys-Asp-Val-Gly-Ile-Gln-Val-Asp- | (SEQ ID NO: 14) |
| Arg-Asp, | |
| Lys-Asp-Thr-Gly-Ile-Gln-Thr-Asp- | (SEQ ID NO: 15) |
| Arg-Asp, | |
| Lys-Asp-Thr-Gly-Ile-Gln-Val-Cys- | (SEQ ID NO: 16) |
| Arg-Asp, | |
| Lys-Asp-Thr-Gly-Ile-Gln-Val-Asn- | (SEQ ID NO: 17) |
| Arg-Asp, | |
| Lys-Asp-Thr-Gly-Ile-Gln-Val-Asp- | (SEQ ID NO: 18) |
| Leu-Asp, | |
| Lys-Asp-Thr-Gly-Ile-Gln-Val-Asp- | (SEQ ID NO: 19) |
| Cys-Asp, | |
| Lys-Asp-Thr-Gly-Ile-Gln-Val-Asp- | (SEQ ID NO: 20) |
| Phe-Asp, | |
| Lys-Asp-Thr-Gly-Ile-Gln-Val-Asp- | (SEQ ID NO: 21) |
| Tyr-Asp, | |
| Lys-Asp-Thr-Gly-Ile-Gln-Val-Asp- | (SEQ ID NO: 22) |
| Arg-Phe, | |
| Lys-Asp-Thr-Gly-Ile-Gln-Val-Asp- | (SEQ ID NO: 23) |
| Arg-Trp, | |
| Val-Asp-Thr-Gly-Ile-Gln-Val-Asp- | (SEQ ID NO: 24) |
| Arg-Tyr, | |
| Ile-Asp-Val-Gly-Ile-Gln-Val-Asp- | (SEQ ID NO: 25) |
| Trp-Trp, | |
| Ile-Asp-Val-Gly-Ile-Gln-Thr-Asp- | (SEQ ID NO: 26) |
| Trp-Asp, | |
| Ile-Asp-Val-Gly-Ile-Gln-Thr-Asp- | (SEQ ID NO: 27) |
| Trp-Trp, | |
| Ile-Asp-Val-Gly-Ile-Gln-Thr-Cys- | (SEQ ID NO: 28) |
| Trp-Trp, | |
| Cys-Asp-Thr-Gly-Ile-Gln-Val-Asp- | (SEQ ID NO: 29) |
| Trp-Asp, | |
| Ile-Asp-Thr-Gly-Ile-Gln-Val-Asp- | (SEQ ID NO: 30) |
| Trp-Asp, | |
| Val-Asp-Thr-Gly-Ile-Gln-Val-Asp- | (SEQ ID NO: 31) |
| Trp-Asp, | |
| Lys-Asp-Val-Gly-Ile-Gln-Val-Asp- | (SEQ ID NO: 32) |
| Trp-Asp, | |
| Cys-Asp-Val-Gly-Ile-Gln-Val-Asp- | (SEQ ID NO: 33) |
| Trp-Asp, | |
| Val-Asp-Val-Gly-Ile-Gln-Val-Asp- | (SEQ ID NO: 34) |
| Trp-Asp, | |
| Cys-Asp-Ile-Gly-Ile-Gln-Val-Asp- | (SEQ ID NO: 35) |
| Trp-Asp, | |
| Ile-Asp-Ile-Gly-Ile-Gln-Val-Asp- | (SEQ ID NO: 36) |
| Trp-Asp, | |
| Val-Asp-Ile-Gly-Ile-Gln-Val-Asp- | (SEQ ID NO: 37) |
| Trp-Asp, | |
| Lys-Asp-Phe-Gly-Ile-Gln-Val-Asp- | (SEQ ID NO: 38) |
| Trp-Asp, | |
| Cys-Asp-Phe-Gly-Ile-Gln-Val-Asp- | (SEQ ID NO: 39) |
| Trp-Asp, | |
| Ile-Asp-Phe-Gly-Ile-Gln-Val-Asp- | (SEQ ID NO: 40) |
| Trp-Asp, | |
| Val-Asp-Phe-Gly-Ile-Gln-Val-Asp- | (SEQ ID NO: 41) |
| Trp-Asp, | |
| Cys-Asp-Glu-Gly-Ile-Gln-Val-Asp- | (SEQ ID NO: 42) |
| Trp-Asp, | |
| Ile-Asp-Glu-Gly-Ile-Gln-Val-Asp- | (SEQ ID NO: 43) |
| Trp-Asp, | |
| Val-Asp-Glu-Gly-Ile-Gln-Val-Asp- | (SEQ ID NO: 44) |
| Trp-Asp, | |
| Lys-Asp-Cys-Gly-Ile-Gln-Val-Asp- | (SEQ ID NO: 45) |
| Trp-Asp, | |
| Cys-Asp-Cys-Gly-Ile-Gln-Val-Asp- | (SEQ ID NO: 46) |
| Trp-Asp, | |
| Ile-Asp-Cys-Gly-Ile-Gln-Val-Asp- | (SEQ ID NO: 47) |
| Arg-Asp, | |
| Val-Asp-Cys-Gly-Ile-Gln-Val-Asp- | (SEQ ID NO: 48) |
| Arg-Asp, | |
| His-Asp-Val-Gly-Ile-Gln-Val-Asp- | (SEQ ID NO: 49) |
| Trp-Asp, | |
| Ser-Asp-Val-Gly-Ile-Gln-Val-Asp- | (SEQ ID NO: 50) |
| Trp-Asp, | |
| Thr-Asp-Val-Gly-Ile-Gln-Val-Asp- | (SEQ ID NO: 51) |
| Trp-Asp, | |
| Lys-Glu-Val-Gly-Ile-Gln-Val-Asp- | (SEQ ID NO: 52) |
| Trp-Asp, | |
| Lys-Asp-Ile-Gly-Ile-Gln-Val-Asp- | (SEQ ID NO: 53) |
| Trp-Asp, | |
| Lys-Asp-Glu-Gly-Ile-Gln-Val-Asp- | (SEQ ID NO: 54) |
| Trp-Asp, | |
| Lys-Asp-Gln-Gly-Ile-Gln-Val-Asp- | (SEQ ID NO: 55) |
| Trp-Asp, | |
| Lys-Asp-Val-Ala-Ile-Gln-Val-Asp- | (SEQ ID NO: 56) |
| Trp-Asp, | |
| Lys-Asp-Val-Gly-Val-Gln-Val-Asp- | (SEQ ID NO: 57) |
| Trp-Asp, | |
| Lys-Asp-Val-Gly-Thr-Gln-Val-Asp- | (SEQ ID NO: 58) |
| Trp-Asp, | |
| Lys-Asp-Val-Gly-Ile-Gln-Val-Asp- | (SEQ ID NO: 59) |
| Ile-Asp, | |
| Lys-Asp-Val-Gly-Ile-Gln-Val-Asp- | (SEQ ID NO: 60) |
| Trp-Glu, | |
| Ala-Ile-Glu-Pro-Val-Leu-Ser-Ile- | (SEQ ID NO: 61) |
| Leu-Asn, | |
| Arg-Ile-Glu-Pro-Val-Leu-Ser-Ile- | (SEQ ID NO: 62) |
| Leu-Asn, | |
| Asn-Ile-Glu-Pro-Val-Leu-Ser-Ile- | (SEQ ID NO: 63) |
| Leu-Asn, | |
| Asp-Ile-Glu-Pro-Val-Leu-Ser-Ile- | (SEQ ID NO: 64) |
| Leu-Asn, | |
| Gln-Ile-Glu-Pro-Val-Leu-Ser-Ile- | (SEQ ID NO: 65) |
| Leu-Asn, | |
| Gly-Ile-Glu-Pro-Val-Leu-Ser-Ile- | (SEQ ID NO: 66) |
| Leu-Asn, | |
| Pro-Ile-Glu-Pro-Val-Leu-Ser-Ile- | (SEQ ID NO: 67) |
| Leu-Asn, | |
| Ser-Ile-Glu-Pro-Val-Leu-Ser-Ile- | (SEQ ID NO: 68) |
| Leu-Asn, | |
| Thr-Ile-Glu-Pro-Val-Leu-Ser-Ile- | (SEQ ID NO: 69) |
| Leu-Asn, | |
| Glu-Phe-Glu-Pro-Val-Leu-Ser-Ile- | (SEQ ID NO: 70) |
| Leu-Asn, | |
| Glu-Ile-Asn-Pro-Val-Leu-Ser-Ile- | (SEQ ID NO: 71) |
| Leu-Asn, | |
| Glu-Ile-Asp-Pro-Val-Leu-Ser-Ile- | (SEQ ID NO: 72) |
| Leu-Asn, | |
| Glu-Ile-Cys-Pro-Val-Leu-Ser-Ile- | (SEQ ID NO: 73) |
| Leu-Asn, | |
| Glu-Ile-Gln-Pro-Val-Leu-Ser-Ile- | (SEQ ID NO: 74) |
| Leu-Asn, | |
| Glu-Ile-Glu-Ala-Val-Leu-Ser-Ile- | (SEQ ID NO: 75) |
| Leu-Asn, | |
| Glu-Ile-Glu-Arg-Val-Leu-Ser-Ile- | (SEQ ID NO: 76) |
| Leu-Asn, | |
| Glu-Ile-Glu-Asn-Val-Leu-Ser-Ile- | (SEQ ID NO: 77) |
| Leu-Asn, | |
| Glu-Ile-Glu-Asp-Val-Leu-Ser-Ile- | (SEQ ID NO: 78) |
| Leu-Asn, | |
| Glu-Ile-Glu-Gln-Val-Leu-Ser-Ile- | (SEQ ID NO: 79) |
| Leu-Asn, | |
| Glu-Ile-Glu-Glu-Val-Leu-Ser-Ile- | (SEQ ID NO: 80) |
| Leu-Asn, | |
| Glu-Ile-Glu-Gly-Val-Leu-Ser-Ile- | (SEQ ID NO: 81) |
| Leu-Asn, | |
| Glu-Ile-Glu-His-Val-Leu-Ser-Ile- | (SEQ ID NO: 82) |
| Leu-Asn, | |
| Glu-Ile-Glu-Lys-Val-Leu-Ser-Ile- | (SEQ ID NO: 83) |
| Leu-Asn, | |
| Glu-Ile-Glu-Met-Val-Leu-Ser-Ile- | (SEQ ID NO: 84) |
| Leu-Asn, | |
| Glu-Ile-Glu-Ser-Val-Leu-Ser-Ile- | (SEQ ID NO: 85) |
| Leu-Asn, | |
| Glu-Ile-Glu-Thr-Val-Leu-Ser-Ile- | (SEQ ID NO: 86) |
| Leu-Asn, | |
| Glu-Ile-Glu-Pro-Ile-Leu-Ser-Ile- | (SEQ ID NO: 87) |
| Leu-Asn, | |
| Glu-Ile-Glu-Pro-Val-Pro-Ser-Ile- | (SEQ ID NO: 88) |
| Leu-Asn, | |
| Glu-Ile-Glu-Pro-Val-Leu-Ala-Ile- | (SEQ ID NO: 89) |
| Leu-Asn, | |
| Glu-Ile-Glu-Pro-Val-Leu-Val-Ile- | (SEQ ID NO: 90) |
| Leu-Asn, | |
| Glu-Ile-Glu-Pro-Val-Leu-Ser-Leu- | (SEQ ID NO: 91) |
| Leu-Asn, | |
| Glu-Ile-Glu-Pro-Val-Leu-Ser-Phe- | (SEQ ID NO: 92) |
| Leu-Asn, | |
| Glu-Ile-Glu-Pro-Val-Leu-Ser-Trp- | (SEQ ID NO: 93) |
| Leu-Asn, | |
| Glu-Ile-Glu-Pro-Val-Leu-Ser-Tyr- | (SEQ ID NO: 94) |
| Leu-Asn, | |
| Glu-Ile-Glu-Pro-Val-Leu-Ser-Val- | (SEQ ID NO: 95) |
| Leu-Asn, | |
| Glu-Ile-Glu-Pro-Val-Leu-Ser-Ile- | (SEQ ID NO: 96) |
| Leu-Ala, | |
| Glu-Ile-Glu-Pro-Val-Leu-Ser-Ile- | (SEQ ID NO: 97) |
| Leu-Asp, | |
| Glu-Ile-Glu-Pro-Val-Leu-Ser-Ile- | (SEQ ID NO: 98) |
| Leu-Gln, | |
| Glu-Ile-Glu-Pro-Val-Leu-Ser-Ile- | (SEQ ID NO: 99) |
| Leu-Glu, | |
| Glu-Ile-Glu-Pro-Val-Leu-Ser-Ile- | (SEQ ID NO: 100) |
| Leu-Gly, | |
| Glu-Ile-Glu-Pro-Val-Leu-Ser-Ile- | (SEQ ID NO: 101) |
| Leu-His, | |
| Glu-Ile-Glu-Pro-Val-Leu-Ser-Ile- | (SEQ ID NO: 102) |
| Leu-Met, | |
| Glu-Ile-Glu-Pro-Val-Leu-Ser-Ile- | (SEQ ID NO: 103) |
| Leu-Pro, | |
| Glu-Ile-Glu-Pro-Val-Leu-Ser-Ile- | (SEQ ID NO: 104) |
| Leu-Ser, | |
| Glu-Ile-Glu-Pro-Val-Leu-Ser-Ile- | (SEQ ID NO: 105) |
| Leu-Thr, | |
| or | |
| Glu-Ile-Glu-Asp-Val-Leu-Ser-Phe- | (SEQ ID NO: 106) |
| Leu-Gly. | |

25. A nucleic acid molecule encoding a protein, a protein fragment, or a peptide of claim 23 or claim 24, said nucleic acid having the nucleotide sequence SEQ ID NO:1.

26. A pharmaceutical composition comprising a protein or protein fragment of claim 23, or a molecule with the following formula: 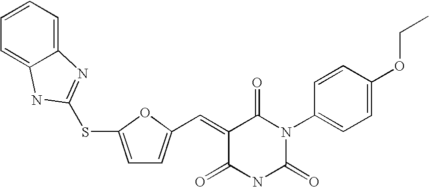
in association with an acceptable pharmaceutical vehicle.

27. A method of treating prediabetes, hyperinsulinemia, or type II diabetes comprising administration of a protein or protein fragment of claim 23, or a molecule with the following formula:

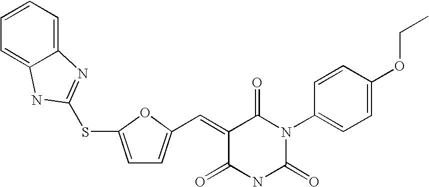

28. A kit for detecting a compound that reduces complexation between the PIN and nNOS that contains the following:
the pancreatic form of nNOS,
PIN,
media or buffers needed for dilution,
materials needed for washing, as necessary
media or buffers needed for the formation of a complex between the PIN and nNOS, and the formation of a complex between the PIN or the nNOS and the compound submitted to the detection procedure, and
means to detect variation, specifically a decrease in the quantity of complex formed with the nNOS or with the PIN.

29. A method of preventing or treating prediabetes, hyperinsulinemia, or type II diabetes in a patient, said method comprising administering to the patient a compound that interferes with the binding of nNOS to PIN.
